(19) United States
(12) Patent Application Publication
(10) Pub. No.: US 2007/0100222 A1
(43) Pub. Date: May 3, 2007

(54) Analyte sensing apparatus for hospital use

(75) Inventors: John J. Mastrototaro, Los Angeles, CA (US); Rajiv Shah, Rancho Palos Verdes, CA (US); Partha Ray, Simi Valley, CA (US); Kenny J. Long, Simi Valley, CA (US); Andrew C. Hayes, Simi Valley, CA (US); Nandita Patel, Los Angeles, CA (US); Cary D. Talbot, Santa Clarita, CA (US); Bahar Reghabi, Marina Del Rey, CA (US)

Correspondence Address: PILLSBURY WINTHROP SHAW PITTMAN LLP, P.O BOX 10500, McLean, VA, 22102, US

(73) Assignee: METRONIC MINIMED, INC., Northridge, CA (US)

(21) Appl. No.: 11/633,254

(22) Filed: Dec. 4, 2006

Related U.S. Application Data
(63) Continuation-in-part of application No. 10/867,529, filed on Jun. 14, 2004.

(51) Int. Cl. A61B 5/00; G06Q 10/00

(52) U.S. Cl. 600/365; 128/903; 128/904; 705/2

(57) ABSTRACT

A system is provided for monitoring blood glucose data of a patient. The system includes a sensing device and hospital monitor. The sensing device includes a sensor and sensor electronics and is adapted to transmit information to the hospital monitor while continuing to sense blood glucose data. The communication between the sensing device and the hospital monitor may be wireless. The sensor electronics may include a sensor power supply, a voltage regulator, and optionally a memory and processor.

### RELATED APPLICATION DATA

This application is a continuation-in-part of U.S. patent application Ser. No. 10/867,529, entitled “System for Providing Blood Glucose Measurements to an Infusion Device,” filed Jun. 4, 2004, the contents of which are herein incorporated by reference.

### BACKGROUND OF THE INVENTION

1. Field of the Invention

This invention relates generally to an analyte sensor for hospital use. More specifically, this invention relates to an analyte sensor that interacts with hospital monitors.

2. Description of Related Art

Over the years, a variety of implantable electrochemical sensors have been developed for detecting and/or quantifying specific agents or compositions in a patient's blood. For instance, glucose sensors have been developed for use in obtaining an indication of blood glucose levels in a diabetic patient. Such readings are useful in monitoring and/or adjusting a treatment regimen which typically includes the regular administration of insulin to the patient. Thus, blood glucose readings improve medical therapies with semi-automated medication infusion pumps of the external type, as generally described in U.S. Pat. Nos. 4,562,751; 4,678,408; and 4,685,903; or automated implantable medication infusion pumps, as generally described in U.S. Pat. No. 4,573,994, which are herein incorporated by reference. While the term “analyte” is used herein, it is possible to determine and use other characteristics as well in the same type of system.

Patients with Type 1 diabetes and some patients with Type 2 diabetes use insulin to control their blood glucose (BG) level. Diabetics must modify their daily lifestyle to keep their body in balance. To do so, diabetics need to keep strict schedules, including ingesting timely nutritious meals, partaking in exercise, monitoring BG levels daily, and adjusting administering insulin dosages accordingly. Testing of BG levels has been both painful and awkward for the patient. Traditionally, insulin dependent diabetics were required to monitor their BG levels by puncturing a finger tip with a needle. Due to the fact that many patients must conduct such a test multiply times throughout the day to regulate their BG levels, the procedure can be painful and inconvenient.

Typically, patients may employ various calculations to determine the amount of insulin to inject. For example, bolus estimation software is available for calculating an insulin bolus. Patients may use these software programs on an electric computing device, such as a computer, the Internet, a personal digital assistant (PDA), or an insulin deliver device. Insulin delivery devices include infusion pumps, injection pens, and implantable delivery systems. The better bolus estimation software takes into account the patient's present BG level. Presently, a patient must measure his/her blood glucose using a BG measurement device, such as a test strip meter, a continuous glucose measurement system, or a hospital hemacue. BG measurement devices use various methods to measure the BG level of a patient, such as a sample of the patient's blood, a sensor in contact with a bodily fluid, an optical sensor, an enzymatic sensor, or a fluorescent sensor. When the BG measurement device has generated a BG measurement, the measurement is displayed or stored in the BG measurement device. Then the patient may visually read the BG measurement and physically enter the BG measurement into an electronic computing device to calculate a bolus estimate. Finally, once the bolus estimate is calculated, the patient must inject the insulin bolus or program into an insulin delivery device to deliver the bolus into the body.

A significant number of diabetic patients still prefer not to use infusion pump devices. These patients may be intimidated by the complex technology or wary of the control of the infusion device. Others may not be able to afford the costs associated with these devices. Such patients may continue to use multiple daily injections (MDI) to administer their insulin dosages. These patients may still benefit from an analyte sensor that can help them monitor analytes such as blood glucose.

In hospitals, patients often need a number of analytes and other physiological characteristics monitored. They may be monitored by sensors that are connected to hospital monitors with displays, which may be able to display a number of characteristics at the same time. Patients often need to move from hospital room to hospital room, which may require an entirely new sensor to be placed in the patient at each room (or a movement of equipment from one room to another).

Medical sensing systems designed to measure a physiological characteristic of a patient generally consist of a sensor and a user interface for setting up the sensor and observing data from the sensor. Typically, the sensor requires power, which is supplied by the user interface or by electronics that accompany the sensor on the user's body. In some environments, it is inconvenient for a person to wear the sensor and the accompanying electronics or user interface, especially if the electronics are large such as a wall mounted display. For example, in a hospital, it is common to have patient monitors that display data about patients, such as heart rate, blood pressure and the like. If a sensor is in communication with a patient monitor, it may be needed or desired to remove the sensor. Yet, the patient cannot always remove the sensor as needed or desired, especially if the sensor is difficult to remove or if the sensor is a single use device, which must be replaced with a new sensor each time it is removed. Thus, new systems are needed that allow the patient to wear the sensor continuously, without the constant inconvenience of a user interface.

### BRIEF SUMMARY

Embodiments of the invention are directed to a sensing device for monitoring blood glucose comprising a blood glucose sensor to sense blood glucose data of a patient and sensor electronics adapted to communicate with a hospital monitor. The sensing device transmits device information to the hospital monitor and is capable of transmitting the information while remaining connected to the patient. The device information may include patient information, such as a patient identification number. The device information may also include sensor information, such as a sensor identification number or sensor and/or calibration history. Communication between the hospital monitor and the sensing device may be wired or wireless.

In further embodiments, the transmission of device information is automatic when a request is received from the hospital monitor for device information. In further embodiments, where communication is wired, transmission of information between the hospital monitor and the sensing device may begin when the wired connection is made.

In further embodiments, the sensing device may periodically, such as once every few minutes or seconds or continuously, transmit via a wireless method a ready communication indicating that it is ready to communicate with a hospital monitor. When the hospital monitor receives a ready communication from a sensing device, it transmits a request for information to the sensing device. In further embodiments, the hospital monitor may be sending out requests for sensing information periodically. When a sensing device comes within reception area of the transmission, it may transmit the sensing information to the hospital device. The distance that the sensing device needs to be from the hospital monitor before the two devices can communicate may be predetermined.

In embodiments of the invention, the sensing device includes an indicator to indicate that the hospital monitor is requesting information from the sensing device, like a visual flash or an audible beep. In further embodiments, the sensor electronics include a memory for storing blood glucose data sensed by the sensor and/or calibration values. The memory may be nonvolatile, like flash memory. The calibration values may be factory supplied reference values or obtained from a blood glucose meter. In embodiments of the invention, a blood glucose meter is provided in the hospital monitor and/or the sensing device to provide calibration data to the sensing device.

### BRIEF DESCRIPTION OF THE DRAWINGS

A detailed description of embodiments of the invention will be made with reference to the accompanying drawings, wherein like numerals designate corresponding parts in the figures.

FIG. 1A is a communication flow diagram of a sensor and user interface in accordance with an embodiment of the present invention.

FIG. 1B is a communication flow diagram of a sensor and user interface and auxiliary device in accordance with an embodiment of the present invention.

FIG. 1C is a communication flow diagram of a sensor and user interface and auxiliary devices in accordance with an embodiment of the present invention.

FIG. 1D is a communication flow diagram of a sensor and user interface and auxiliary device in accordance with an embodiment of the present invention.

FIG. 1E is a communication flow diagram of a sensor and user interface and auxiliary device in accordance with an embodiment of the present invention.

FIG. 1F is diagram of an embodiment of the present invention in accordance with the information flow diagram of FIG. 1B.

FIG. 1G is diagram of an embodiment of the present invention in accordance with the information flow diagram of FIG. 1B.

FIG. 1H is diagram of an embodiment of the present invention in accordance with the information flow diagram of FIG. 1c.

FIG. 2A is an information flow diagram of a sensor, sensor electronics, and user interface in accordance with an embodiment of the present invention.

FIG. 2B is an information flow diagram of a sensor, sensor electronics, user interface and display device in accordance with an embodiment of the present invention.

FIG. 2C is an information flow diagram of a sensor, sensor electronics, user interface, and display devices in accordance with an embodiment of the present invention.

FIG. 2D is an information flow diagram of a sensor, sensor electronics, user interface, and display device in accordance with an embodiment of the present invention.

FIG. 2E is an information flow diagram of a sensor, sensor electronics, user interface, and display device in accordance with an embodiment of the present invention.

FIG. 2F is diagram of an embodiment of the present invention in accordance with the information flow diagram of FIG. 2B.

FIG. 2G is diagram of an embodiment of the present invention in accordance with the information flow diagram of FIG. 2B.

FIG. 2H is diagram of an embodiment of the present invention in accordance with the information flow diagram of FIG. 2B.

FIG. 2I is diagram of an embodiment of the present invention in accordance with the information flow diagram of FIG. 2B.

FIG. 2J is diagram of an embodiment of the present invention in accordance with the information flow diagram of FIG. 2C.

FIG. 2K is diagram of an embodiment of the present invention in accordance with the information flow diagram of FIG. 2C.

FIG. 2L is diagram of an embodiment of the present invention in accordance with the information flow diagram of FIG. 2D.

FIG. 2M is diagram of an embodiment of the present invention in accordance with the information flow diagram of FIG. 2D.

FIG. 2N is diagram of an embodiment of the present invention in accordance with the information flow diagram of FIG. 2D.

FIG. 2O is diagram of an embodiment of the present invention in accordance with the information flow diagram of FIG. 2D.

FIG. 2P is diagram of an embodiment of the present invention in accordance with the information flow diagram of FIG. 2E.

FIG. 2Q is diagram of an embodiment of the present invention in accordance with the information flow diagram of FIG. 2E.

FIG. 2R is diagram of an embodiment of the present invention in accordance with the information flow diagram of FIG. 2E.

FIG. 2S is diagram of an embodiment of the present invention in accordance with the information flow diagram of FIG. 2E.

FIG. 3A shows a sensor in accordance with an embodiment of the present invention.

FIG. 3B shows a sensor with incorporated electronics in accordance with an embodiment of the present invention.

FIG. 3C shows a sensor connected with a previously separate sensor electronics that includes a wire for connecting to another device in accordance with an embodiment of the present invention.

FIG. 4A shows a sensor connected to a previously separate sensor electronics including a transmitter in accordance with an embodiment of the present invention.

FIG. 4B shows a sensor connected to a previously separate sensor electronics including a transmitter in accordance with an embodiment of the present invention.

FIG. 4C shows a sensor and electronics encased in a housing which includes a transmitter in accordance with an embodiment of the present invention.

FIG. 5A is a block diagram of a user interface and sensor in accordance with an embodiment of the present invention.

FIG. 5B is a block diagram of a user interface, auxiliary device and sensor in accordance with an embodiment of the present invention.

FIGS. 5C and 5D are block diagrams of a user interface, sensor and sensor electronics in accordance with embodiments of the present invention.

FIGS. 5E and 5F are block diagrams of a user interface, sensor and sensor electronics in accordance with embodiments of the present invention.

FIG. 5G is a block diagram of a user interface, sensor and sensor electronics in accordance with an embodiment of the present invention.

FIG. 5H is a block diagram of a user interface, sensor and sensor electronics in accordance with an embodiment of the present invention.

FIGS. 6A and 6B are block diagrams of a user interface, sensor and sensor electronics in accordance with embodiments of the present invention.

FIGS. 6C and 6D are block diagrams of a user interface, sensor and sensor electronics in accordance with embodiments of the present invention.

FIG. 6E is a block diagram of a user interface, sensor and sensor electronics in accordance with an embodiment of the present invention.

FIG. 7 is a diagram of an electronics architecture according to an embodiment of the invention with a custom integrated circuit.

FIG. 8 is a data flow chart of a sensor and hospital monitor in accordance with an embodiment of the present invention.

### DETAILED DESCRIPTION

In the following description, reference is made to the accompanying drawings, which form a part hereof and which illustrate several embodiments of the present inventions. It is understood that other embodiments may be utilized and structural and operational changes may be made without departing from the scope of the present inventions.

As shown in the drawings for purposes of illustration, the invention may be embodied in a physiological characteristic sensing system including a physiological characteristic sensor, such as a blood glucose sensor, that generates physiological characteristic data to be sent to one or more devices, such as a user interface and/or an auxiliary device. The physiological characteristic data may be displayed on the auxiliary device.

An auxiliary device according to the present invention may be a hospital monitor. For example, some patient monitors are used in a hospital environment to monitor physiological characteristics of a patient, such as the patient monitors described in U.S. Pat. No. 6,733,471, hereby incorporated by reference. A hospital monitor according to the present invention may include a display, one or more input devices, such as keypads, remotes, touch screens, microphones, or the like, and a receiver. The receiver may be a wired receiver, and receive information from sensors wired to the monitor, or a wireless receiver, which would receive information from sensors over wireless frequencies. The receiver may alternatively be adapted to receive wired and wireless information from sensors. The monitor may further be adapted to receive one or more modules that allow for it to interact with particular sensors. For example, the blood pressure data coming from a blood pressure cuff may be adapted to transmit to hardware that is not necessarily in the monitor. The hardware could be put on the module, which would then be inserted into the monitor if the user wanted the monitor to receive and show blood pressure information.

In the hospital situation, a number of factors make a traditional home-use analyte sensor inadequate. For example, a home-use analyte sensor is generally adapted to only be used with one monitor. So, when the sensor is calibrated, it is calibrated using that particular monitor and is used with that monitor throughout its life. A sensor that is wired to a portable monitor is also not generally adapted to plug into a hospital monitor, because the wire is short for convenience of the user. For wireless sensors, a hospital environment can be an unsuitable environment. It is common for a number of patients to be in the same room, which means that even with standard precautions, it is more likely for sensors transmitting data to interfere with each other. In addition, the patients often move from room to room. Because the sensors are only adapted to interact with one monitor, there would be a complicated setup involved each time a patient moved to a new room.

Physiological characteristics are generally used in a hospital to detect when a patient needs a therapy change and to quantify the therapeutic change required. For example, a patient's blood glucose level may be measured to determine if they have lost metabolic control. If they have lost metabolic control, a caregiver can use the blood glucose measurement to determine changes to therapy. Hospital patients may lack metabolic control due to trauma, stress of surgery, stroke, heart conditions, myocardial infarction, hypertension, diabetes, organ transplant, infections, sepsis, renal diseases, pregnancy, physical, mental or emotional distress, and the like.

In accordance with embodiments of the invention, an analyte sensor is provided that allows for easy and convenient measurement of a patient's analyte levels, such as, for example, blood glucose (BG) levels. In embodiments where the analyte sensor is a BG sensor, the included features can be tailored for use with patients of all types, such as multiple daily injection (MDI) users as well as infusion device users. Furthermore, the BG sensor can be used with any variety of therapy/diagnostic devices, such as medication infusion devices, electronic therapy devices, and devices that receive diagnostic information from cardiac and other sensors. Some examples include, but are not limited to, an external or internal infusion pump, an injection pen, an intravenous (IV) drip, or an inhaler for an inhalable drug such as inhalable insulin.

In other embodiments, lactate sensors may be used to detect a patient's blood lactate concentration. Lactate concentrations can be used to detect whether a patient has had a myocardial infarction or whether a patient is septic. Rising lactate levels can indicate that a patient is becoming more septic, and lowering lactate levels can indicate that a patient is recovering from sepsis. Lactate levels may also be used to determine the how efficiently a patient's tissue is using oxygen. As the tissue oxygen exchange decreases, the lactate level increases, and caregivers can detect that the patient is becoming more ill.

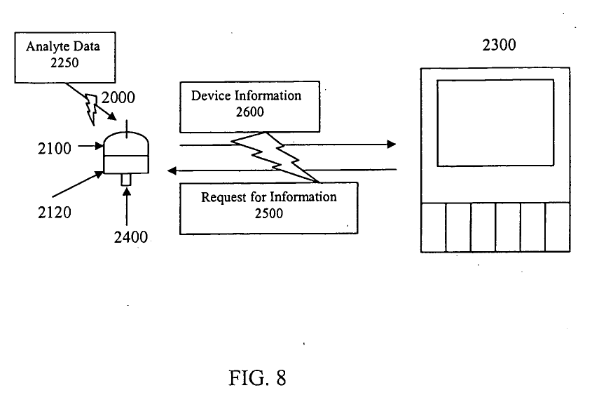

In embodiments according to the present invention, an analyte sensor is adapted to exchange information with one or more hospital auxiliary devices, such as hospital monitors. As shown in FIG. 8, in an embodiment of the invention, an analyte sensor 2000 is adapted to communicate with a hospital monitor 2300. The analyte sensor may communicate through wired or wireless communication. The wireless methods include, by no way in limitation, RF, infrared (IR), Bluetooth, ZigBee, and other 802.15 protocol, 802.11 WiFi, spread spectrum communication, and frequency hopping communication. Embodiments that use multiple frequencies can facilitate better communication because the sensor can continually switch frequencies until it finds the strongest frequency in the area with which to communicate. For example, a chip may allow the sensor to do the scanning of the frequencies and then to frequency hop to the strongest signal. In embodiments using wireless options, there may be employed a “spread spectrum” where a large range of frequencies can be used to relay the communication. “Frequency hopping,” or changing frequencies to pick up whatever frequency is present, may also be used. Another embodiment is one that uses adaptive frequency selection, or Listen Before Talk (LBT), where the devices select the cleanest available channel from those allotted prior to transmitting. In some cases, frequency hopping allows the system to find frequencies that are not being used by other nearby systems and thus avoid interference. In addition, a system may operate in a manner where each component-to-component communication is on a different frequency, or where the delay for each communication is different. Other types of wireless communication may also be used for communication, such as translation frequency.

In another wireless example, if the user has access to a computer network or phone connection, the user can open communication via the Internet to obtain communications from, and send communications to, various computers over the Internet, such as a nurse or doctor. A transceiver may be used to facilitate data transfer between a personal computer (PC) and the medication device. Such a communication may be also used by a party, other than the user, to control, suspend, and/or clear alarms. In the hospital setting, this may be, for example, a doctor in another room of the hospital. As a non-limiting example, further description of a communication station may be found in U.S. Pat. No. 5,376,070, which is herein incorporated by reference. The transceiver may allow clinicians in a hospital setting to communicate with the various components of the sensor and/or an infusion system wirelessly. The transceiver may be used to download device information from the sensor and/or infusion system to a hospital monitor an/d or personal computer (PC) when the transceiver communicates to that monitor and/or PC. In embodiments, the transceiver may be wired to a hospital monitor and/or PC so that it may derive its power from the monitor and/or PC when the two are connected. In this way, the transceiver conveniently does not require a separate power source.

In wired embodiments, there may be a tether physically connecting the sensor to a user interface or the monitor/PC. In yet further embodiments, the sensor and the medication device may be wired and wireless—when wired, the components communicate by wire, and when disconnected, the components communicate through wireless communication.

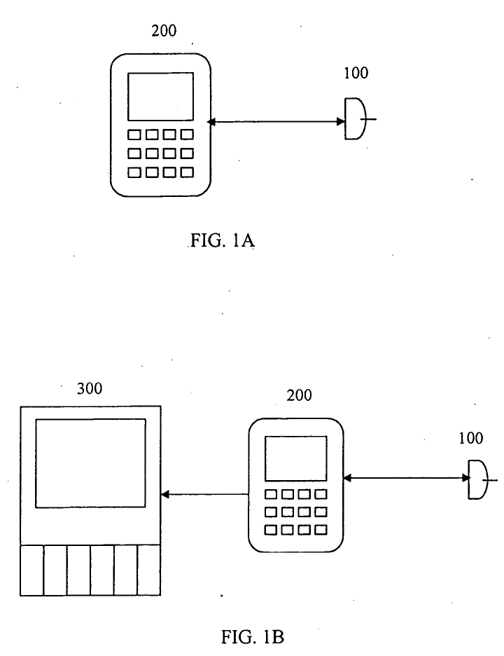

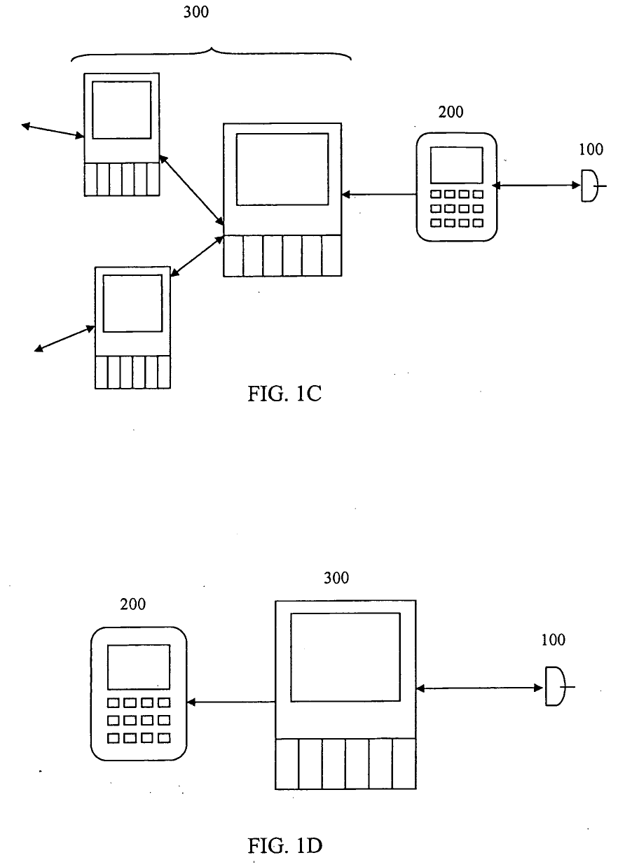

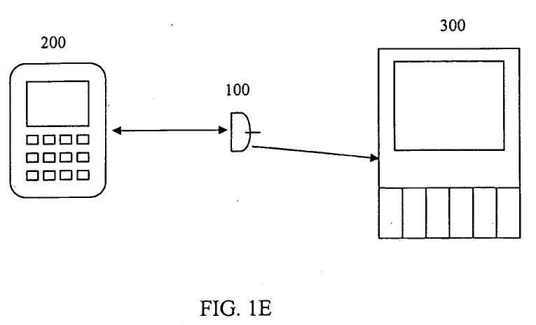

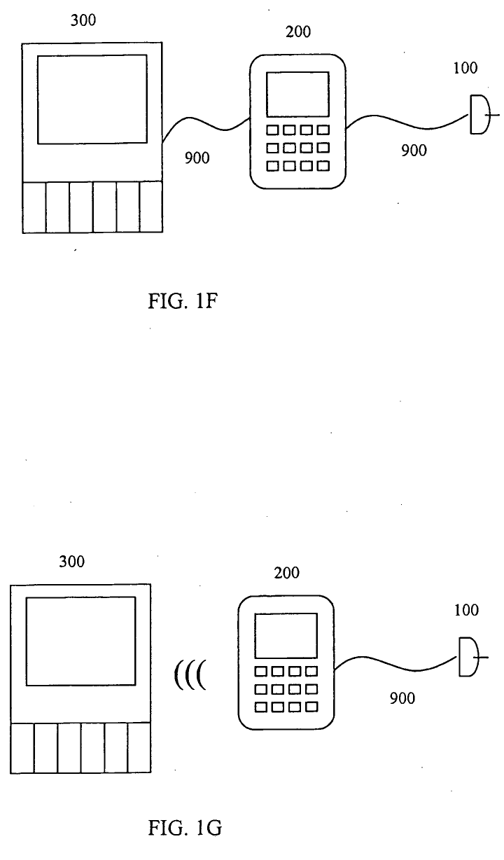

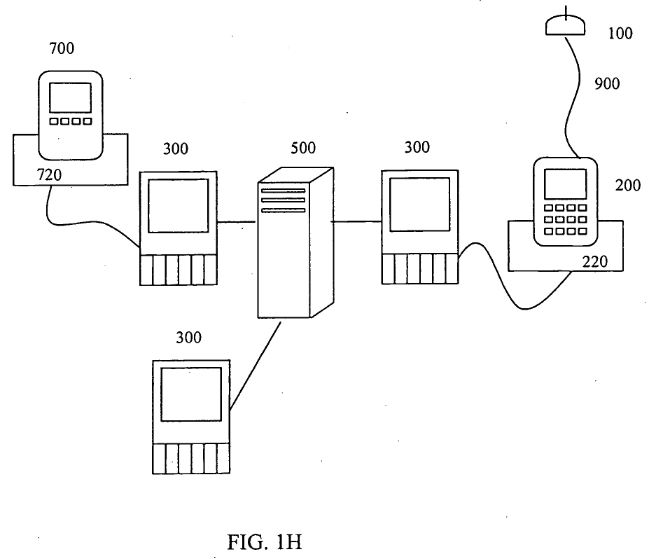

FIGS. 1A-1H show wired connections between a sensor 100 and one or more devices according to embodiments of the present invention. The one or more devices include at least a user interface 200 and may include one or more auxiliary devices 300. There may be a connector between wired components (not shown). As shown in FIG. 1A, the present invention may consist of a sensor 100 in communication with a user interface 200. The sensor 100 is powered by the user interface 200, and the sensor 100 measures a physiological characteristic, such as blood glucose concentration.

The sensor may continuously measure a physiological characteristic, and then measurement updates would be displayed periodically on one or more devices. The sensor measurements may be real-time, and thus would be displayed as soon as the measurement is available. Alternatively, more than one measurement may be collected before a measurement is displayed. The measurements also may be stored until all measurements are taken and then displayed. The measurement may also be delayed before it is displayed.

In embodiments of the invention, the sensor is a subcutaneous sensor (also known as a transcutaneous sensor), which is inserted through the skin of the patient. In further embodiments, the sensor may be another type of sensor, such as an implanted sensor. The sensor may also measure, in addition or in lieu of blood glucose concentration, the concentration of, oxygen, potassium, hydrogen potential (pH), lactate, one or more minerals, analytes, chemicals, proteins, molecules, vitamins, and the like, and/or other physical characteristics such as temperature, pulse rate, respiratory rate, pressure, and the like. The sensor may be an electrochemical sensor placed through skin into the subcutaneous tissue of a body such as the sensor described in U.S. Pat. Nos. 5,390,671, 5,391,250, 5,482,473, and 5,586,553, and U.S. patent application Ser. No. 10/273,767 (published as U.S. patent publication no. 2004/0074785 A1, Apr. 22, 2004), which are herein incorporated by reference. Alternatively, the sensor may be a blood contacting sensor. For example, the sensor may be a thin film vascular sensor such as described in U.S. Pat. Nos. 5,497,772, 5,660,163, 5,750,926, 5,791,344, 5,917,346, 5,999,848, 5,999,849, 6,043,437, 6,081,736, 6,088,608, 6,119,028, 6,259,937, 6,472,122, and 6,671,554, and U.S. patent application Ser. No. 10/034,627 (published as U.S. patent publication no. 2003/0078560 A1, Apr. 24, 2003), Ser. No. 10/331,186 (published as U.S. patent publication no. 2004/0061232 A1, Apr. 1, 2004), Ser. No. 10/671,996 (published as U.S. patent publication no. 2004/0061234 A1, Apr. 1, 2004), Ser. No. 10/335,574 (published as U.S. patent publication no. 2004/0064156 A1, Apr. 1, 2004), Ser. No. 10/334,686 (published as U.S. patent publication no. 2004/0064133 A1, Apr. 1, 2004), and Ser. No. 10/365,279 (published as U.S. patent publication no. 2003/0220552 A1, Nov. 27, 2003), which are herein incorporated by reference. Alternatively, the sensor may be non-invasive and thus, does not penetrate into the body such as optical sensors and the sensor described in U.S. patent application Ser. No. 09/465,715, (published as PCT application no. US99/21703, Apr. 13, 2000), which is herein incorporated by reference. The sensor may preferably be a real-time sensor. As used herein, the terms “real-time” and “real-time sensor” refer to a sensor that senses values substantially continuously over an extended period of time and makes such values available for use as the values are being sensed and collected rather than having to download substantially all the collected values at a later time for use. For example, a real-time blood glucose sensor might sense glucose values every 10 seconds over an extended period of 24 hours, and make the values available (e.g., processing, charting and displaying) every 5 minutes so that that users of an insulin pump have the flexibility to fine-tune and start or stop insulin delivery upon demand. Patients may thus use their pumps to make substantially immediate therapy adjustments based upon real-time continuous glucose readings displayed every 5 minutes and by viewing a graph with 24-hour glucose trends. For example, the sensor may be as described in U.S. patent application Ser. No. 10/141,375 (published as U.S. patent publication no. 2002/0161288 A1, Oct. 31, 2002), hereby incorporated by reference, and the view of displayed data may be as described in U.S. patent application Ser. No. 10/806,114, which is herein incorporated by reference.

In preferred embodiments, sensor measurements are displayed every 5 minutes. Alternatively they may be displayed more frequently such as every 2 minutes, every minute, or every 30 seconds. In other embodiments the sensor value is displayed less frequently such as every 7 minutes, 8 minutes, 10 minutes, 15 minutes, 20 minutes, 30 minutes, 1 hour, and the like. Periodically a nurse may observe a patient's present blood glucose level and adjust the patient's therapy such as changing the insulin delivery rate (e.g., increasing or decreasing the rate that a pump supplies insulin to the patient's body through intravenous or subcutaneous delivery), providing an extra bolus of insulin (e.g., injecting extra insulin into the patient's body, or into the patient's IV line, or by programming an insulin pump to infuse an extra dose of insulin), change the patient's food intake (e.g., increasing or decreasing the rate that glucose is delivered into the patient's body, or changing the rate of tube feeding, or giving the patient food to consume), changing the amount of drugs that the patient is using that affect insulin activity such as medications to treat type 2 diabetes, steroids, anti-rejection drugs, antibiotics, and the like. The nurse might check the patient's glucose level and make an adjustment to therapy as needed every hour. Alternatively, a nurse may see if an adjustment is needed more frequently such as every 30 minutes, 20 minutes, 10 minutes and the like. This is especially likely if the patient's glucose level is not in a normal range. Alternatively a nurse may see if an adjustment is needed less frequently such as every 2 hours, 3 hours, 4 hours, 6 hours and the like. This is more likely if the patient's glucose level is in the normal range; or, if the patient's glucose has been normal for a period such as 1 hour, 2 hours, 4 hours, or 8 hours; or if the patient's therapy has not changed for a period such as 2 hours, 4 hours, 8 hours or 12 hours. In further alternatives, nurses may rely on alarms to notify them to check on the patient. For example, nurses might rely on glucose alarms to tell them that glucose levels are too high or too low before they see if a therapy adjustment is needed, they might rely on an alarm to tell them that it is time to calibrate the sensor, they might rely on a time activated alarm to tell them that it is time to check in on a patient, they might rely on an alarm to tell them that the equipment needs to be cared for, and the like.

A normal range for a patient's blood glucose level in the hospital is typically between 80 and 120 milligrams of glucose per deciliter of blood (mg/dl). Some caregivers maintain a higher normal range with the upper limit of the range at about 140 mg/dl, 145 mg/dl, 150 mg/dl, 160 mg/dl, and the like and the lower limit of the range at about 70 mg/dl, 80 mg/dl, 90 mg/dl, 100 mg/dl, 110 mg/dl, and the like. Other caregivers maintain a lower normal range with the upper limit of the range at about 110 mg/dl, 100 mg/dl, 90 mg/dl, 80 mg/dl, and the like and the lower limit of the range at about 80 mg/dl, 70 mg/dl, 60 mg/dl, 50 mg/dl, and the like.

A caregiver may use the present blood glucose value to adjust a patient's therapy to bring the patient's glucose to within a normal range. For example, if the patient's glucose level is higher than the higher end of the normal range, the caregiver may increase the rate that insulin is delivered to the patient's body. Conversely, if the patient's glucose level is below the lower end of the normal range, the caregiver may decrease the insulin delivery rate.

Alternatively, the caregiver may consider both the present and at least one older glucose value to determine adjustments to the patient's therapy. For example, if the present glucose level is too high and a previous glucose level was lower, then the caregiver may substantially increase the insulin rate because the patient's glucose is too high and rising.

The caregiver may use trend information or a graphical plot of glucose values over time to determine if the patient's therapy should be changed. Alternatively, the therapy may be changed automatically when the patient's glucose level is drifting out of the normal range.

The user interface 200 allows a user to interact with the sensor. The user interface may include one or more of: an output device such as a liquid crystal display (LCD), a light emitting diode (LED), a touch screen, a dot matrix display, plasma display, alarm, buzzer, speaker, sound maker, voice synthesizer, vibrator, and the like; an input device such as a keypad, one or more buttons, a keyboard, a mouse, a joystick, a radio frequency (RF) receiver, an infrared (IR) receiver, an optical receiver, a microphone, and the like. In further embodiments, a pedometer is included to track how much exercise the user is taking. This exercise amount may be used as an external factor to consider in calculating the bolus amount. The user interface may be a handheld device such as a handheld computer, a personal digital assistant (PDA), a cell phone or other wireless phone, a remote control, and the like. Alternatively, the user interface may be a personal computer (PC), a desk top computer, a lap top computer, and the like.

Among other advantages, embodiments of the present invention may provide convenience and ease of use. For example, an embodiment with a user interface and display on the analyte sensor may cater to the active lifestyles of many insulin dependent diabetics. A large and simple display minimizes the potential for error in reading and interpreting test data. A small overall size permits discretion during self-monitoring and makes it easy to carry. In some embodiments, the sensor may include a dedicated backlight to facilitate viewing. The backlight may be a user programmable multi-color backlight that additionally performs the function of a visual indicator by flashing colors appropriate to the level of an alert or alarm. The backlight may also have variable intensity (automatic or manual) to preserve the battery power and improved viewing.

As shown in FIG. 1B, the user interface 200 may also be in communication with an auxiliary device 300, such as a patient monitor. A patient monitor includes any display or other indicator system intended to be used in a hospital, doctor's office, or other medical setting, including home medical use. For example, some patient monitors are used in a hospital environment to monitor physiological characteristics of a patient, such as the patient monitors described in U.S. Pat. No. 6,733,471, hereby incorporated by reference.

Although the arrow from the user interface 200 is shown transmitting data to auxiliary device 300 and not in reverse, this is not in any way intended to be limiting. In any of the figures shown, the transmission of data may occur in either, or both, directions. The communication may be over a wired connection or by wireless methods. Wireless methods include methods such as radio frequency (RF) communication, infrared (IR) communication, optical communication or any other wireless method that would be useful in connection with the present invention as would be readily appreciated by one of ordinary skill in the art without undue experimentation. In further embodiments, the sensor or user interface may further include a retractable antenna on the housing for increasing reception or strength of frequency.

As shown in FIG. 1C, the user interface 200 may communicate with one or more auxiliary devices 300. The one or more auxiliary devices 300 may communicate with each other in addition to the user interface 200 and/or the sensor 100 directly.

As shown in FIG. 1D, the sensor 100 may be in communication directly with the auxiliary device 300. The user interface 200 thus may communicate with the auxiliary device 300 which may communicate with the sensor 100. Additionally, as shown in FIG. 1E, the sensor 100 may communicate both with the user interface 200 and with the auxiliary device 300.

FIGS. 1F and 1G illustrate arrangements of embodiments of the present invention in accordance with the data flow of FIG. 1B. As shown in FIG. 1F, the sensor 100 may be tethered to the user interface 200 by a wire 900, and the user interface 200 may be tethered to the auxiliary device 300 by a wire 900. As shown in FIG. 1G, even if the sensor 100 is tethered to the user interface 200 by a wire 900, the user interface 200 may communicate wirelessly with the auxiliary device 300.

One or more of the auxiliary devices may be in communication with a personal computer or server, so that sensor measurements are sent to the personal computer or server. As shown in FIG. 1H, one or more of the auxiliary devices 300 may be in communication with a personal computer or server 500, and blood glucose (BG) reference measurements from a BG meter 700 or a laboratory measurement are sent to the personal computer. In further embodiments a BG meter may be integrated into the user interface or sensor or in the auxiliary device (e.g., a patient monitor). In such embodiments, a receptacle is provided in the housing of the device for receiving and testing a fluid sample from the user to determine the concentration of blood glucose in the user. A test strip that may hold a fluid sample is inserted into the receptacle for the testing. In variations, there may be a cartridge-like mechanism which loads and presents the strip for testing and then ejects it. In further embodiments, a lancing device may be provided and coupled to the receptacle for directly obtaining the sample without a test strip. Reference measurements may be sent to a personal computer or server 500, and then sent to the user interface 200. These reference measurements may be used for calibration of the sensor data. As shown in FIG. 1H, the user interface 200 may communicate with the personal computer or server 500 through one or more other auxiliary devices 300, such as a patient monitor. The communication with the BG meter 700 and the user interface 200 may also be through one or more of the auxiliary devices 300. Also as shown in FIG. 1H, the user interface 200 may communicate through a docking station 220. The BG meter 700 may also be placed in a docking station 720. The sensor measurements may be stored on a server and made available to one or more PCs. Thus in one example, sensor information can be downloaded to a first PC, the BG meter reference measurements can be downloaded or entered into a second PC, the first PC and the second PC can communicate with each other (such as through a server), the reference measurements can be sent to the user interface, and the sensor measurements and/or reference measurements can be viewed at any of the PCs that are connected to the shared server. One or more devices, such as the user interface and/or the BG meter may use one or more cradles to connect the device to a PC. Alternatively, the reference measurements are sent to a PC, the processed sensor signal is sent to a PC, and the PC calculates the sensor measurements. Alternatively, the user interface may communicate with a personal computer using radio frequency (RF) (not shown). Examples of devices to facilitate communication with the personal computer include, without limitation, communications linking devices such as the ComLink™ sold by Medtronic MiniMed, IR cradles, RF devices, or the like that can be used to send and/or receive signals. For example, the ComLink™ has a transceiver to receive RF signals from a user interface and then forwards received information to the personal computer by wire.

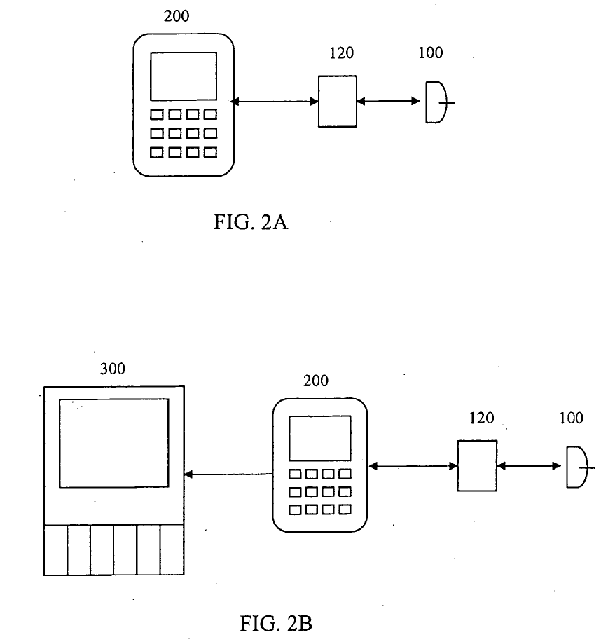

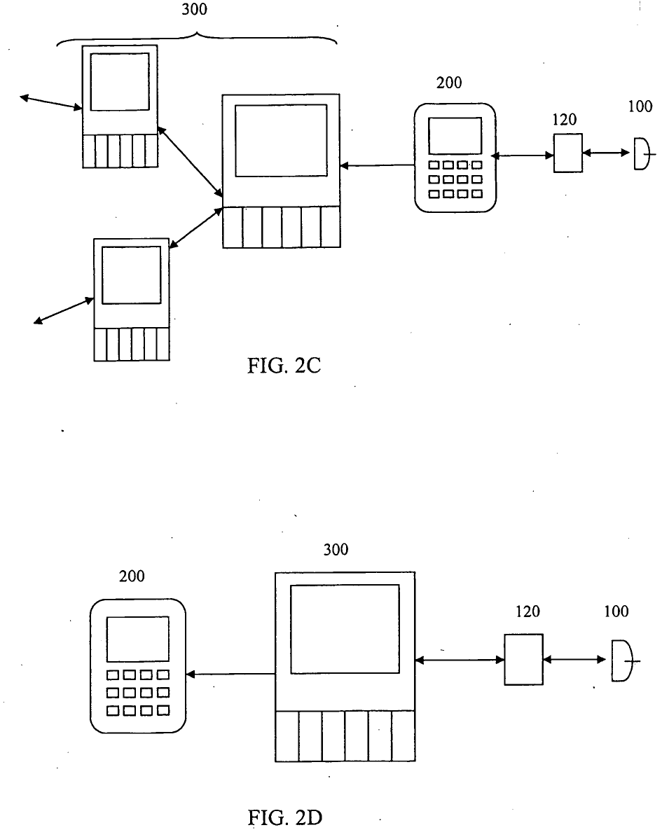

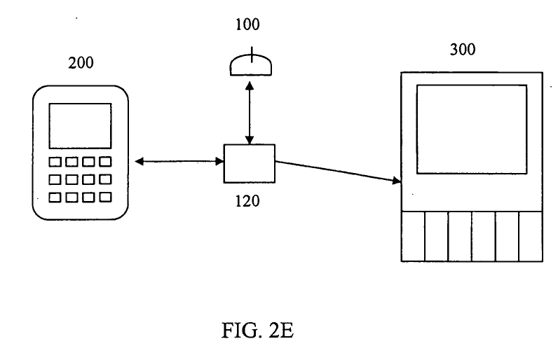

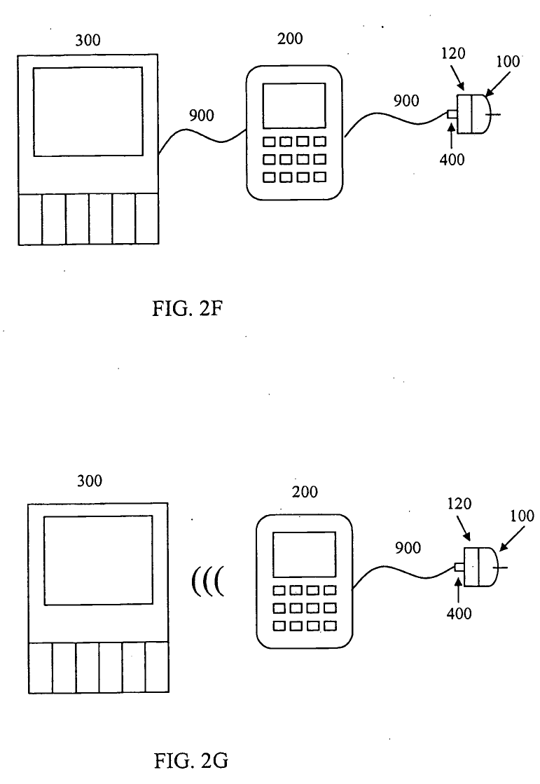

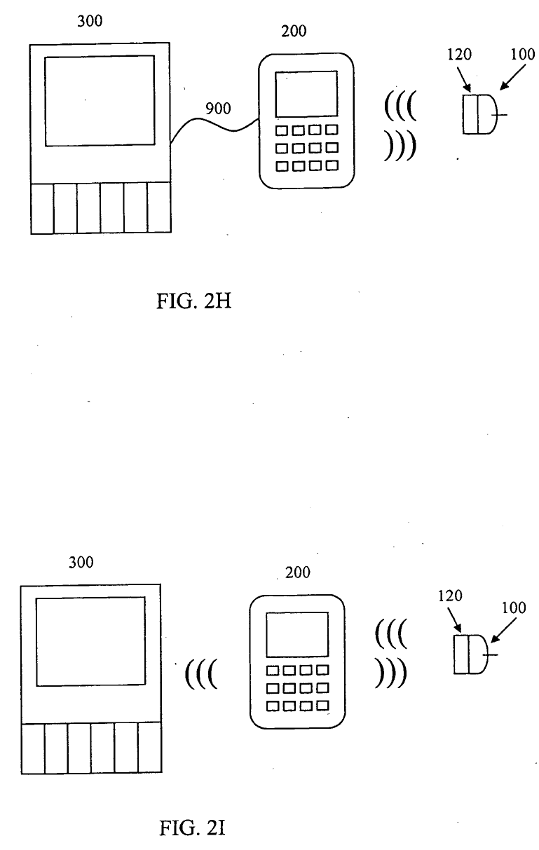

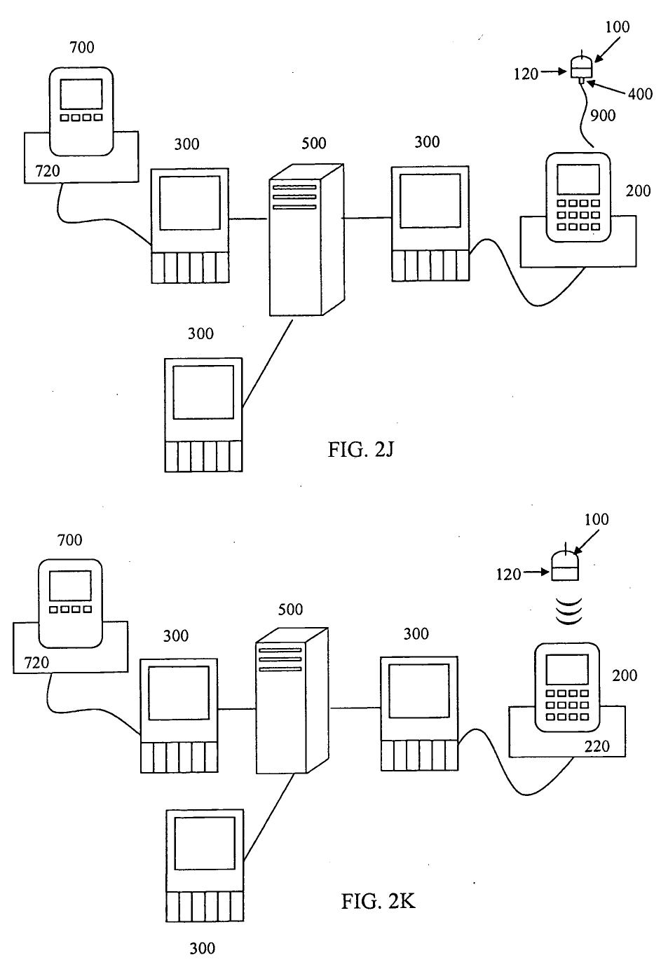

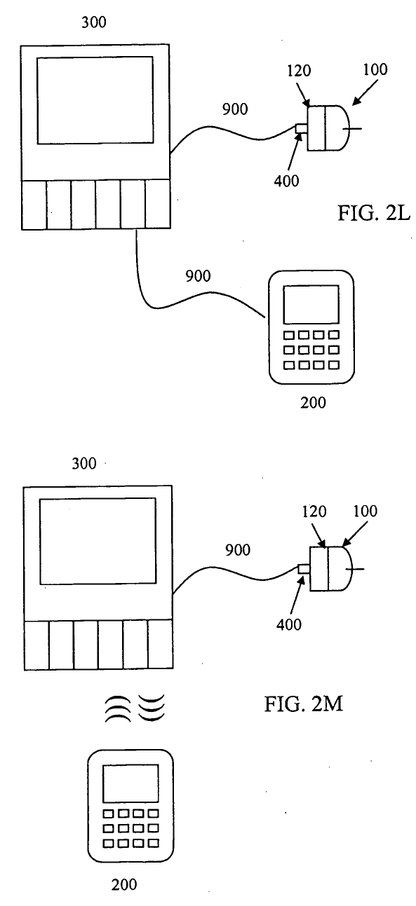

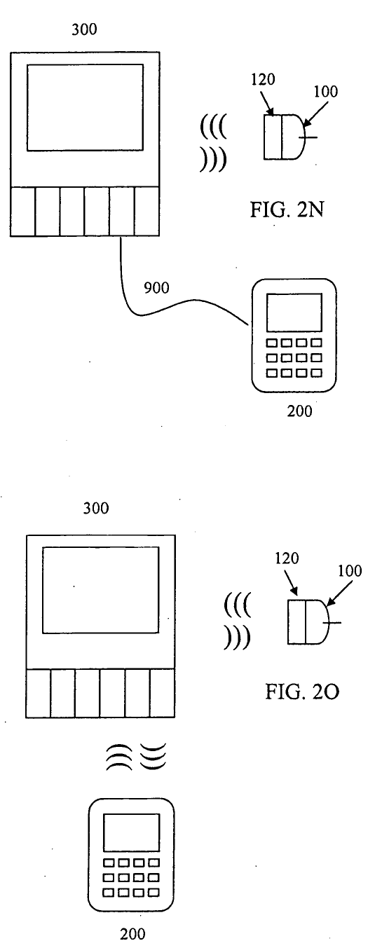

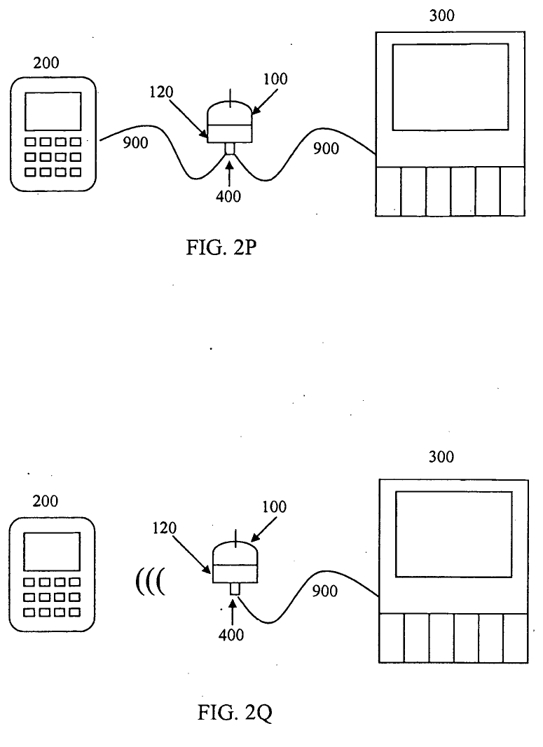

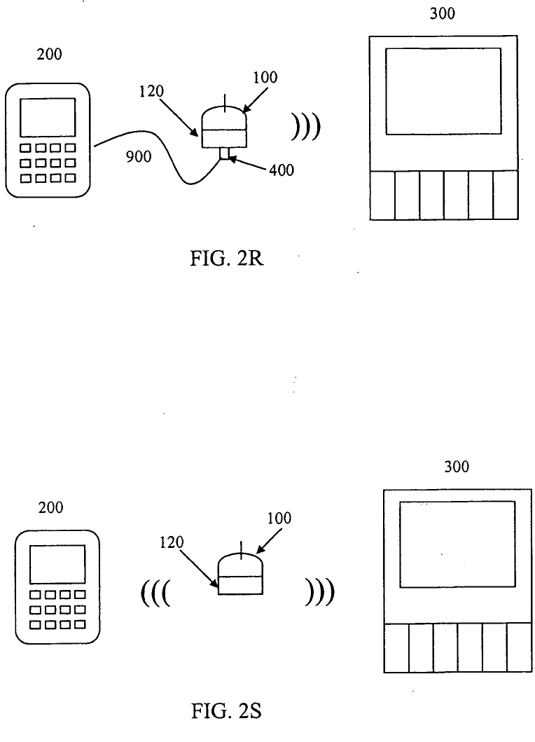

FIGS. 2A-2S show data flow of embodiments of the present invention where a sensor communicates with sensor electronics, which communicate to a user interface. The sensor is tethered to sensor electronics, which may communicate over a tethered connection or wirelessly to a user interface and/or auxiliary device. A more detailed discussion of the sensor electronics is included below. As shown in FIG. 2A, a sensor 100 may be in communication with sensor electronics 120, which are in communication with the user interface 200.

In FIG. 2B, the user interface 200 is in communication with one or more auxiliary devices 300, as well as in communication with the sensor electronics 120. As shown in FIG. 2C, the user interface 200 may be in communication with more than one auxiliary device 300. The auxiliary devices 300 may be in communication with each other and/or in communication with the user interface 200 and/or sensor electronics 120.

As shown in FIG. 2D, both the user interface 200 and the sensor electronics 120 may communicate with the auxiliary device 300. And as shown in FIG. 2E, the sensor electronics 120 may be in communication with both the user interface 200 and the auxiliary device 300.

FIGS. 2F-2I, 2L-2O, and 2P-2S are embodiments of the present invention in accordance with the data flow of FIGS. 2B, 2D, and 2E, respectively. They illustrate that the communications between devices may be by wire 900 or may be wireless. In FIGS. 2F and 2G, the sensor 100 and sensor electronics 120 are coupled to each other and to a connector 400. The connector 400 may connect the sensor electronics 120 to a wire 900 that connects to the user interface 200. As shown in FIG. 2F, the user interface 200 may then be tethered to an auxiliary device 300 via a wire 900. As shown in FIG. 2G, the user interface 200 may also be in wireless communication with the auxiliary device 300.

In FIGS. 2H and 2I, the sensor 100 and sensor electronics 120 are coupled to each other but communicate wirelessly to the user interface 200. There need not be a connector in this embodiment, but it is possible to have a sensor and sensor electronics that can communicate through wired or wireless configurations to the user interface. Therefore, the sensor and sensor electronics may be coupled to a wire connector that is not in use when the communication is wireless. In FIGS. 2H and 2I, the sensor 100 is coupled to the sensor electronics 120, which is in wireless communication with the user interface 200. As shown in FIG. 2H, the user interface 200 may then be tethered to an auxiliary device 300 via a wire 900. As shown in FIG. 2I, the user interface 200 may also be in wireless communication with the auxiliary device 300.

In FIGS. 2L and 2M, the sensor 100 and sensor electronics 120 are coupled to each other and to a connector 400. The connector 400 may connect the sensor electronics 120 to a wire 900 that connects to the auxiliary device 300. As shown in FIG. 2L, the auxiliary device 300 may then be tethered to a user interface 200 via a wire 900. As shown in FIG. 2M, the auxiliary device 300 may also be in wireless communication with the user interface 200.

In FIGS. 2N and 2O, the sensor 100 and sensor electronics 120 are coupled to each other but communicate wirelessly to the auxiliary device 300. In FIGS. 2N and 2O, the sensor 100 is coupled to the sensor electronics 120, which is in wireless communication with the auxiliary device 300. As shown in FIG. 2N, the auxiliary device 300 may then be tethered to a user interface 200 via a wire 900. As shown in FIG. 2O, the auxiliary device 300 may also be in wireless communication with the user interface 200.

In FIGS. 2P, 2Q and 2R, the sensor 100 and sensor electronics 120 are coupled to each other and to a connector 400. The connector 400 may couple the sensor electronics 120 to one or more wires 900 that connects to the auxiliary device 300 and/or the user interface 200. As shown in FIG. 2P, the sensor electronics 120 may be coupled to both auxiliary device 300 and user interface 200 via wires 900. As shown in FIG. 2Q, the sensor electronics 120 may be coupled to the auxiliary device 300 via wire 900 and in wireless communication with the user interface 200. As shown in FIG. 2R, the sensor electronics 120 may be coupled to the user interface 200 via wire 900 and in wireless communication with the auxiliary device 300. In FIG. 2S, the sensor 100 is coupled to the sensor electronics 120, which is in wireless communication with the auxiliary device 300 and with the user interface 200.

One or more of the auxiliary devices may be a personal computer or server, and sensor measurements may be sent to the personal computer or server. Additionally, blood glucose (BG) reference measurements from a BG meter or a laboratory measurement may be sent to the personal computer or server, and then may be sent to the user interface. As shown in FIGS. 2J and 2K, the user interface 200 may communicate with a personal computer 500, and a BG meter 700 may communicate with the personal computer 500. Also as shown in FIGS. 2J and 2K, the user interface 200 may communicate with the personal computer or server 500 through one or more other auxiliary devices 300, such as a patient monitor. The communication with the BG meter 700 and the user interface 200 may also be through one or more of the auxiliary devices 300. The user interface 200 may communicate through a docking station 220. The BG meter 700 may also be placed in a docking station 720. In FIG. 2J the sensor 100 is coupled to the sensor electronics 120, which is coupled to a connector 400 for coupling the sensor electronics 120 to the user interface through a wire 900. As shown in FIG. 2K, the communication between the sensor electronics 120 (coupled to the sensor 100) and the user interface 200 may also be wireless. The sensor information may be stored on a server and made available to one or more personal computers. Thus in one example, sensor information can be downloaded to a first personal computer, the BG meter reference measurements can be downloaded or entered into a second personal computer, the first personal computer and the second personal computer can communicate with each other (such as through a server), the reference measurements can be sent to the user interface, and the sensor measurements and/or reference measurements can be viewed at any of the personal computers that are connected to the shared server. Alternatively, the reference measurements may be sent to a personal computer, the processed sensor signal may be sent to a personal computer, and the personal computer may then calculate the sensor measurements.

As discussed above, the present invention may include electrical components. For example, the electrical components may include one or more power supplies, regulators, signal processors, measurement processors, reference memories, measurement memories, user interface processors, output devices, and input devices. The one or more power supplies provide power to the other components. The regulator supplies regulated voltage to one or more sensors, and at least one of the one or more sensors generates a sensor signal indicative of the concentration of a physiological characteristic being measured. Then the signal processor processes the sensor signal generating a processed sensor signal. Then the measurement processor calibrates the processed sensor signal using reference values from the reference memory, thus generating sensor measurements. Then the measurement memory stores sensor measurements. Finally, the sensor measurements are sent to the user interface processor, which forwards the sensor measurements to an output device.

The one or more power supplies may be a battery. Alternatively, the one or more power supplies may be one or more batteries, a voltage regulator, alternating current from a wall socket, a transformer, a rechargeable battery, or the like. The regulator may be a voltage regulator. Alternatively, the regulator may be a current regulator, or other regulator. The source of power for operating the sensor or for charging a battery within sensor electronics may include an AC power source (e.g., 110-volt or 220-volt), DC power source (e.g., a 12-volt DC battery), or pulsating DC power source (e.g., a power charger that provides pulsating DC current to a battery that re-energizes the battery and removes the lead sulfate deposits from the plates). The battery may be a single use or a rechargeable battery. Where the battery is rechargeable, there may be a connector or other interface on a device to attach the device to an electrical outlet, docking station, portable recharger, or so forth to recharge the battery while in the device. It is also possible that a rechargeable battery may be removable from the device for recharging outside of the device, however, in some cases, the rechargeable battery may be sealed into the housing of the device to create a more water resistant or waterproof housing. The devices may be adapted to accommodate various battery types and shapes. In embodiments, the devices may be adapted to accommodate more than one type of battery. For example, a device may be adapted to accommodate a rechargeable battery and, in the event of battery failure or other need, also adapted to accommodate a readily available battery, such as an AA battery, AAA battery, or coin cell battery.

n an embodiment of the present invention, the processor of the medication device uses power cycling such that power is periodically supplied to the communication system of the medication device until a communication is received from the sensor, for example, a BG sensor. When a communication is received from the sensor, the processor of the medication device discontinues using power cycling so that the power is continuously supplied to the medication device communication system. The medication device processor may then resume using power cycling upon completing the receipt of the communication including the data indicative of the determined concentration of the analyte in the user from the sensor communication system.

The signal processor may perform one or more functions such as, converting the sensor signal from an analog signal to a digital signal, clipping, summing, filtering, smoothing, and the like.

The measurement processor may perform one or more functions such as, but not limited to, calibrating (converting the processed sensor signal into measurements), scaling, filtering, clipping, summing, smoothing, analyzing, and the like. The measurement processor may also analyze whether the sensor is generating signals indicative of a physiological characteristic or whether the sensor is no longer functioning properly. For example, the measurement processor may detect that the processed sensor signal is too high, too low, changes too rapidly, or is too noisy for a properly functioning sensor, and thus indicate that the sensor should be replaced. The measurement processor may further analyze whether to generate an alarm due to a characteristic of the sensor measurement, such as the sensor measurement is too high, too low, increasing too rapidly, decreasing too rapidly, increasing too rapidly given its present value, decreasing too rapidly given its present value, too high for a given duration, too low for a given duration, and the like. Additionally, the measurement processor may estimate the remaining battery life.

The reference memory may contain one or more reference values for converting the processed sensor signal into a sensor measurement. For example, 1 micro-amp (μamp) equals 40 milligrams of glucose per deciliter of fluid (mg/dl), or 2 nano-amps equals 10 millimoles of glucose per liter of fluid (mmol/l). Reference measurements are input into the input device periodically during the life of the sensor, with each reference measurement paired with a processed sensor signal, and each pair of a reference measurement with a processed sensor signal stored in the reference memory as a reference value. Thus, the measurement processor may use new reference values to convert the processed sensor signal into sensor measurements. Alternatively, the reference values may be factory installed. Thus no periodic reference measurements are needed. Additionally, the reference memory may contain both factory installed reference values and periodic reference values.

The user interface processor may transfer sensor measurements from the measurement memory to the output device. The user interface processor may also accept inputs from the input device. If the sensor includes a memory, the user interface may send parameters from the inputs to the sensor for storage in the memory. The inputs may include one or more of certain setup parameters, which it may be possible to change later but may be fixed: one or more high thresholds, one or more low thresholds, one or more trend rates, alarm acknowledge, minimum time between alarms, snooze duration, sensor serial number, codes, identification numbers (ID), password, user name, patient identification, reference measurements, and the like. The user interface processor may also tell the output device what to do including one or more of the following: display the latest sensor measurement, display the latest reference measurement, display a graph of sensor measurements, display thresholds, activate an alarm, display a message such as an alarm message, an error message, a command, an explanation, a recommendation, a status, and the like. Additionally, the user interface processor may perform one or more processing or analyzing functions such as, calibrating, scaling, filtering, clipping, summing, smoothing, calculating whether the sensor is generating signals indicative of a physiological characteristic or whether the sensor is no longer functioning properly, estimating remaining battery life, determining whether to generate an alarm due to a characteristic of the sensor measurement, and the like. One such system is described and disclosed in U.S. patent application Ser. No. 10/624,177, entitled “System for Monitoring Physiological Characteristics,” which is herein incorporated by reference. In one embodiment, the display can show analyte levels in a variety of ways—as a present analyte level or a graphical depiction of the analyte levels over a period of time.

The display may also provide different visual analyses of the analyte levels over different time periods. Furthermore, the display may mimic the display on the medication device. In certain embodiments, whatever is shown on the display of the infusion device or injection device corresponds to that shown and reflected on the display of the analyte sensor. The display may also display information according to communications sent to it from the infusion device or injection device that corresponds to the sensor. For example, when the last bolus was administered, when the last alarm occurred, when the last finger stick was taken, past trends, all alarms that occurred in a time period, calibrations, meals, exercise, bolus schedules, temporary basal delivery, diagnostic information, and the like. Whenever a bolus is being delivered, the medication device can send a message every time a tenth of a unit, or some specified amount, is delivered, to which the user may monitor via the analyte sensor display. In this manner, the user may more conveniently view what is being processed or acted upon in the medication device without removing or adjusting the medication device to check the medication device. In embodiments, the sensor may include one or more input device(s), such as keys, buttons, and the like, on a keypad so that all, or substantially all, viewing and data entry may be performed on the same device without moving the medication device.

In embodiments, the analyte sensor includes a “bolus estimator” program which allows the sensor to take into account a variety of factors that may affect blood glucose levels of the user which may in turn affect the amount of insulin needed. For example, in one embodiment, the bolus estimator factors in the other medications that the user is ingesting, especially those that will affect glucose sensitivity, such as for example, glucophage. In other embodiments, the bolus estimator will enable the sensor to factor into the insulin dosage what device the insulin is to be administered through because different devices will administer medication differently. Factoring this differential into the dosage is especially important for those patients who use multiple daily injections rather than infusion devices, as their dosages may change depending on the device they select to inject the insulin.

In further embodiments, the sensor may include capabilities such as setting insulin sensitivity and insulin/carbohydrate ratios. This capability allows users to customize settings of the sensor. For example, the bolus estimator may come with educational tools and protocols that will allow a user to set their insulin sensitivity by ingesting specific foods in specific amounts and analyzing how their blood glucose level fluctuates and/or responds to specific amounts of insulin administered. The results from the analysis can be stored into the sensor memory to apply to the user's settings. In addition, the sensor may also store in memory a database of medications, for example, those that affect insulin sensitivity for future reference. This data may be programmed into the sensor and/or downloaded from specific internet sites. The sensor may also be programmed to prompt alerts to the user when a medication that may affect insulin sensitivity is ingested.

The sensor may also have other user prompts. In one embodiment, the sensor prompts the user to report events that help create event markers that can further help gauge the user's sensitivity to various factors. If there is a rapid increase or decrease in blood glucose level, the sensor realizes the change and will prompt the user with a text message or audio message asking “what just happened-did you just exercise?,” “did you just eat?,” “input what you just ate,” and the like. The information inputted by the user will allow the sensor to analyze how the blood glucose level fluctuates or reacts to specific events. Cataloging such events can help user note, for example, how fast insulin or other medications affect blood glucose level or how much certain foods affect blood glucose level. These events may include, but are not limited to, type of food ingested, amount of food ingested, amount of exercise undertaken, type of drug ingested, amount of drug ingested, type of medication device used, time lapse from last bolus administered, and user sensitivity. Recording specific events may allow a physician or caretaker better monitor and manage the patient's diet and dosage schedules. This information may also be communicated to and monitored through a data management software program like CARELINK (sold by Medtronic Minimed, Inc.). Furthermore, the sensor may be able to organize the sensitivity and/or response patterns from these external factors into a chart for easier analysis and calculation of bolus amount.

In embodiments used with data management software, the sensor may undergo periodic uploads of data, for example, in the middle of the night. These uploads may be performed automatically, without any action on the part of the user. The uploads may include data to upgrade or update the sensor from the central data management station. The uploads may also include data sent by a physician or caretaker via a computer network. Alternatively, the uploads may be conducted via a wire connected between the sensor and the source of the uploaded data. The data management software, such as CARELINK, may also incorporate a SMS server so that messages may be delivered in the form of text messages, as in cellular telephones. The sensors may be adapted to recognize whenever they are in the presence of a management station and upload all the data that those sensors do not already have and save the data to a repository.

In embodiments, the sensor and/or user interface may include a basal estimator which helps to take the information generated by the user and/or bolus estimator and calculates the user's basal flow rate and determines the impact, if any, on the insulin dosages. The basal estimator may provide other features such as suggesting how to better use lancets, and equipment.

There also may be some type of positive mechanism for the analyte sensor if the communication between the analyte sensor and the medication device are interrupted. For example, the mechanism may have the analyte sensor stop displaying its graph in a “time-out” phase for the time the medication device screen is absent or no more data is entered by the user for a period of time. In this case, the medication device operates on the last data that the medication device sent to the analyte sensor to display. In an embodiment, the analyte sensor will display an idle screen during the time-out phase and while the communication between the medication device and the analyte sensor is re-established. The idle screen may remain until the next action is selected by the user. After the time-out phase, the user may press a key to start up the communication again. Once a key is pressed, the analyte sensor will process the key data and the screen will be displayed. The analyte sensor may periodically send signals to the medication device and any other peripheral devices to see if those components are still active on the screen.

In alternative embodiments, there will be a positive confirmation requested prior to displaying graphs. For example, the graphs may be shown in bitmap packets (e.g., bit-by-bit), and if the user will be getting a large number of packets of data, for example 15 packets of data, to show the graph, the user may opt not to confirm. The data is passed from the analyte sensor, which is programmed to display the data, to the medication device. The analyte sensor can operate in graphics description language where data is recognized by the analyte sensor as instructing it on which position to put each line or color and the graphics display would handle determining the resolution that the graph would be displayed in. In some embodiments, the graph may be displayed in three-dimensional format.

If one or more electrical components reside in the same device, then one or more of the electrical components may be combined into a single electrical component, such as combining the user interface processor, measurement processor and the signal processor; or combining the measurement memory and the reference memory. Alternatively, the components may be independent despite in which device they reside.

It is possible that a sensor will need to receive regulated power for a defined duration before it can generate a stable signal, in other words it must warm up. And, if regulated power is removed from the sensor, the sensor must warm up again when the power is restored before measurements can be used. Alternatively, it is possible that each time the sensor is warmed up, new reference measurements must be input and paired with a processed sensor signal to create new reference values, which are stored in the reference memory. Reference values are needed to calibrate the processed sensor signal into sensor measurements. Furthermore, periodic reference values may be needed, and if a stable (warmed up) processed sensor signal is not available when a new reference values is needed, then a new reference measurement may have to be collected when the processed sensor signal is available and stable. In the mean time the processed sensor signal cannot be used to generate a sensor measurement. In other words, if it is time for a new reference measurement to maintain calibration and the sensor signal is not available to pair with the new reference measurement, then the sensor loses calibration and will have to be recalibrated when the sensor signal becomes available. It is also possible that more than one reference value will need to be collected before the sensor measurement is considered calibrated.

Calibration data may come from a variety of places, especially in a hospital environment. The data may come from a traditional test strip BG meter, which may be either separate from the sensor or integrated in the sensor. It may also come from laboratory data. For example, a patient's blood may be drawn in a syringe by a nurse and then tested for its blood glucose level. The algorithms stored in the sensor electronics or other calibration device can be suitable altered to take into account the type of calibration data being used. For example, in a hospital environment, blood tests that are run in the laboratory have significantly more lag time than the traditional test strip BG meter. Thus, the calibration data will need to be synchronized with a sensor reading that it is being compared to. In certain embodiments, the sensor device or other calibration device is adapted to receive an indication that blood is being taken for a laboratory test. This indication may be entered via a button, key, or other input device. When the blood glucose level is later input into the sensor/calibration device, it will be compared to the sensor value taken closest to the time of taking the blood. In further embodiments, the sensor may be adapted to display the time of the calibration data. For example, it may be that the sensor is set up to display the current time as the default time of calibration data. The user may scroll the time, or enter a different time, if the current time is not the correct time of the calibration data. In still further embodiments, the user may enter the time elapsed since the time of the calibration data, for example, “20 minutes ago.” In still further embodiments, the sensor is adapted to synchronize its clock with the auxiliary device, which may be synchronized with the various clocks in the hospital. Thus, if the nurse or other person drawing the blood records the time of drawing the blood, it will be consistent throughout the hospital.

There is a possibility, particularly in a hospital environment, that the sensor may be disconnected from the user interface and/or from the patient monitor for extended periods of time. For example, patients are moved between rooms and beds regularly when the may not be connected to any patient monitor (e.g. a surgery patient may move from admission to surgery to recovery, and so forth). In some cases, calibration will be scheduled at particular intervals. When the sensor, coupled to sensor electronics, is disconnected from the user interface and/or patient monitor, one of these intervals may occur. For such a situation, it is useful to have a way to calibrate the sensor and sensor electronics while separated from the user interface and/or patient monitor. For example, the sensor may include a blood glucose (BG) meter to support calibration. The BG meter may be display-free to, for example, reduce excess size and weight. The BG meter included in the sensor would then provide reference values for calibration to the sensor electronics. It is also possible to couple the sensor electronics to a BG meter or to use a wireless connection to the BG meter to receive the reference values.

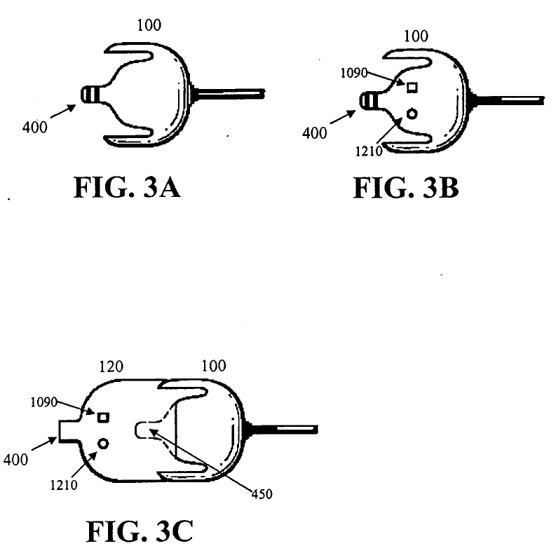

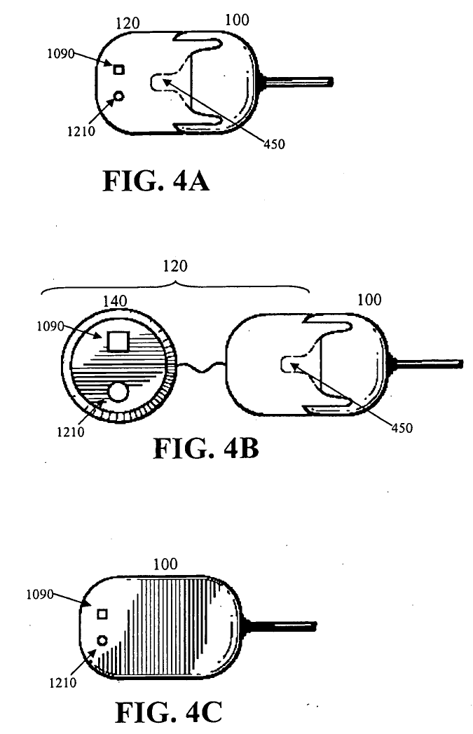

FIGS. 3A-3C and 4A-4C illustrate physical embodiments of aspects of the present invention. FIGS. 3A-3C show sensors with and without sensor electronics with connectors 400, so that they may be wired to one or more devices. In the embodiments shown in FIGS. 1A-1H, discussed above, there is a connector 400 between the sensor 100 and a device, which is not shown. FIG. 3A illustrates a simple sensor in accordance with the invention as embodied in FIGS. 1A-1H. The sensor 100 includes the connector 400. The sensor 100 is not always wired to a device. For example, as shown in FIGS. 3C, 4A, and 4B, the sensor 100 shown in FIG. 3A may be coupled to sensor electronics. In this particular embodiment, however, the sensor 100 does not include sensor electronics.

There are a number of ways to include sensor electronics in the sensor of the present invention. As shown in FIG. 3B, the sensor 100 may include a connector 400 and the sensor electronics may be a monolithic part of the sensor. In FIG. 3B, electrical components, specifically the regulator 1090 and sensor power supply 1210, are shown directly on the sensor 100. Alternatively, the sensor electronics 120 may be coupled to the sensor 100 by a connector 450, such as shown in FIG. 3C. The sensor electronics 120 in FIG. 3C include one or more electrical components, such as the regulator 1090 and sensor power supply 1210 and may be wired to one or more devices through connector 400.

FIGS. 4A-4C show sensors which are intended to be used for wireless communication with one or more devices. As shown in FIG. 4A, the sensor 100 may be coupled to the sensor electronics 120 by a connector 450. The sensor electronics 120 may include one or more electrical components, such as the regulator 1090 and sensor power supply 1210. As shown in FIG. 4B, the sensor may be coupled to a sensor electronics 120 that include a portion coupled to the sensor via a connector 450 and wired to a separate portion 140, which includes sensor electronics. Although the sensor electronics are shown as having electrical components on only one portion, it is possible to have some electrical components on one portion of the sensor electronics and other electrical components on another portion. Embodiments shown in FIG. 4B are discussed in more detail in U.S. patent application Ser. No. 09/465,715, filed Dec. 17, 1999, which is herein incorporated by reference. As shown in FIG. 4C, the sensor electronics may be a monolithic part of the sensor 100.

Many different wireless communication protocols may be used. Some protocols are for one-way communication and others are for two-way communication. For one-way communication, the transmitting device may have a transmitter and the receiving device may have a receiver. For two-way protocols, each device typically has a transceiver, but each device could have a transceiver and a receiver. For any wireless embodiment, a transceiver may be used in place of a receiver or a transmitter, because the transceiver can perform like a receiver or a transmitter or both.

Where the sensor electronics 120 (wired or wireless) are separated from the sensor 100 by a connector 450, such as shown in FIGS. 3C, 4A, and 4B, the sensor electronics may first become powered by the sensor power supply at the time that the sensor electronics are attached to the sensor. Thus, the sensor power supply shelf life is increased. Alternatively, the sensor electronics may always be powered. The sensor electronics may be powered by the sensor power supply when triggered by other means such as, when the user interface is connected to the sensor electronics, when a magnetic switch is triggered, when a mechanical switch is triggered, or the like.

The duty cycle of the sensor power supply may vary based on the sensor electronics being connected or disconnected from the user interface and/or patient monitor. For example, when the sensor electronics are disconnected, the duty cycle may be reduced (e.g., by using fewer electrical components, by decreasing data acquisition, and the like), which will allow for a greater sensor power supply shelf life. If the sensor and sensor electronics lose power for a prolonged period of time, the calibration process may have to be repeated. The sensor electronics may include circuitry to detect low battery levels and may be coupled to an alarm that will activate if the low battery level reaches a certain threshold.

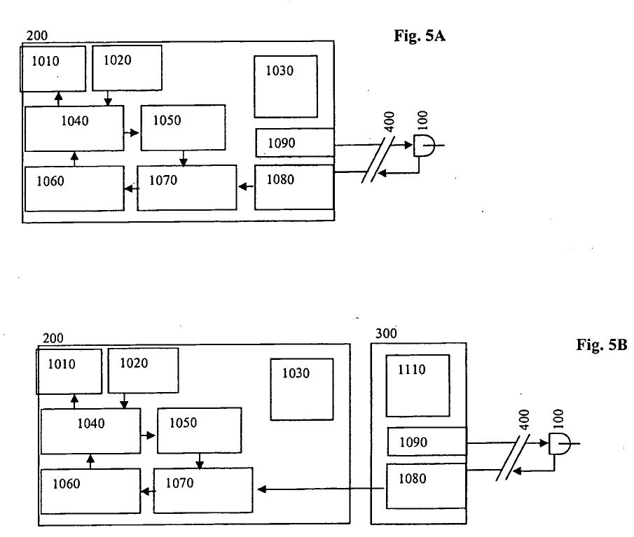

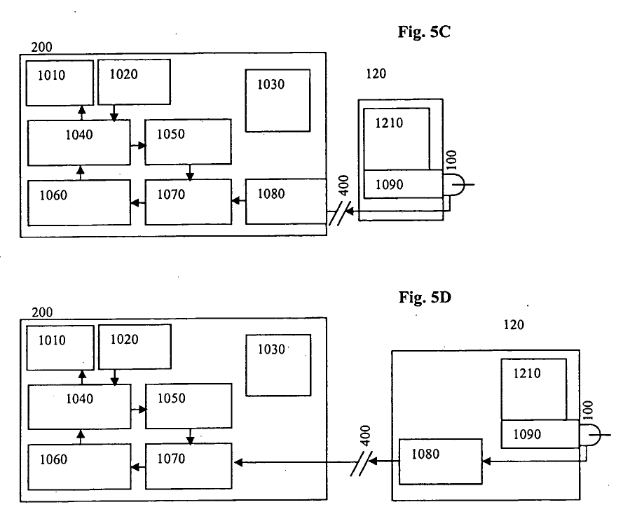

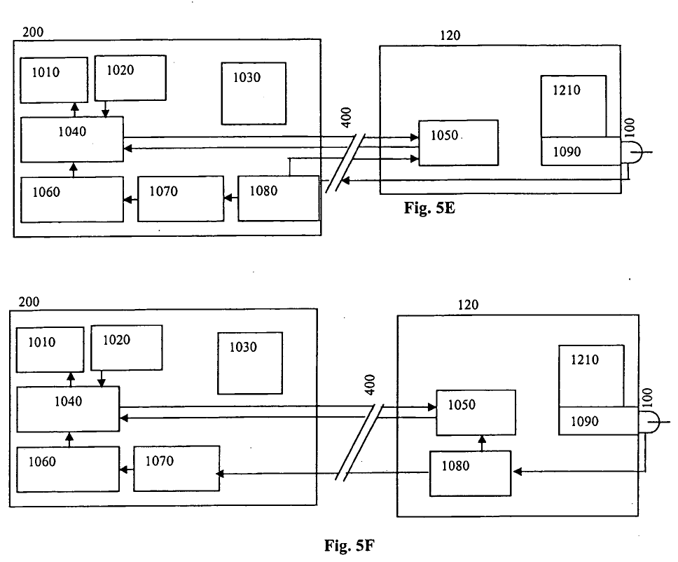

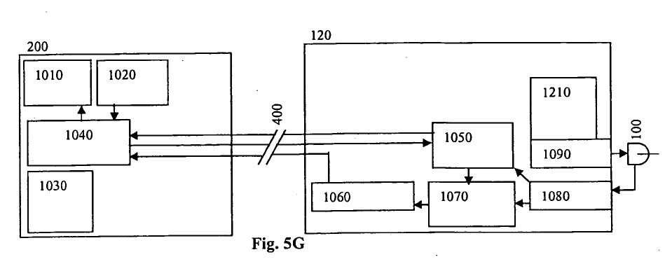

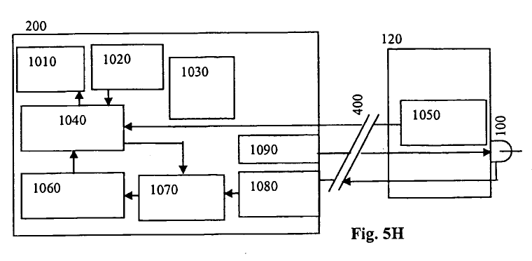

FIGS. 5A-5H are block diagrams of the electronic components of embodiments of aspects of the present invention. In the embodiment shown in FIG. 5A, the user interface 200 is tethered to the sensor 100. The tether may be interrupted by a connector 400 so that the sensor 100 and the user interface 200 can be separated. The sensor 100 does not include a power supply in FIG. 5A. When the patient disconnects a sensor from the user interface 200, then the sensor no longer receives power from the regulator and thus may require time to warm up again and may require re-calibration when re-connected with the user interface.

The user interface power supply 1030 supplies power to the user interface 200 and may also supply power to the sensor 100. The regulator 1090 supplies regulated voltage to sensor 100, and the sensor 100 generates a sensor signal indicative of the concentration of a physiological characteristic being measured. Then the signal processor 1080 processes the sensor signal generating a processed sensor signal. Then the measurement processor 1070 calibrates the processed sensor signal using reference values from the reference memory 1050, thus generating sensor measurements. Then the measurement memory 1060 stores sensor measurements. Finally, the sensor measurements are sent to the user interface processor 1040, which forwards the sensor measurements to an output device 1010. The reference values, and other useful data, may be input through an input device 1020.

As shown in FIG. 5B, an auxiliary device 300 may be tethered to the sensor 100, and the tether may interrupted by a connector 400 so that the sensor 100 and the user interface 200 can be separated. Thus, a patient wearing a sensor does not have to remain tethered to a device, such as a user interface or an auxiliary device. The user can wear the sensor and temporarily or permanently disconnect from other devices. This can be useful if the patient needs to leave the proximity of one or more devices. For example, the sensor may be tethered to a stationary device such as a wall-mounted or bed-mounted display, and the patient must leave the room for a therapeutic procedure. As shown in FIG. 5B, the auxiliary device may include an auxiliary device power supply 1110, regulator 1090 and the signal processor 1080, so that the auxiliary device processes the sensor signal.

In the above embodiments, where the sensor does not include a power supply, when the sensor is disconnected from the other devices, the sensor no longer receives power. The tether includes one or more wires to carry the regulated voltage to the sensor and carry the sensor signal to the signal processor. For particular types of sensors, the sensor must be warmed up again when re-connected with the user interface. Where the reference memory is included in the user interface, one or more reference values may be periodically measured and stored in the reference memory when they are collected. If the sensor is disconnected from the user interface when a new reference value is required, however, the sensor will need calibration when it is re-connected.

One or more devices other than the sensor may be in communication with each other, such as discussed above in reference to FIGS. 1B-1H. The one or more devices other than the sensor, such as an auxiliary device and a user interface, may share a tethered connection such as a wire. As used herein the term “wire” means and includes any physical conductor capable of transmitting information by non-wireless means including, for example, one or more conventional wires, a serial or parallel cable, a fiber optic cable, and the like. The term “wire” also includes any physical conductor capable of carrying regulated voltage, electrical power, and the like. Additionally, the tethered connections may include at least one connector so that at least one device can be separated from the others. One or more of the one or more devices other than the sensor, such as an auxiliary device and a user interface, may communicate wirelessly, such as RF, IR, sub-sonic, and the like communications, such as shown in FIG. 1G.

Alternatively, the user interface may be coupled to sensor electronics, which may be coupled to the sensor, such as shown in FIGS. 5C-5H. If a power supply and regulator stay with the sensor (as part of the sensor electronics), when the sensor is disconnected from the user interface, then the sensor can remain powered and retain calibration. Thus, the sensor may not require warm up time and may not require re-calibration when re-connected to the same user interface that it was connected to previously.

The sensor power supply may be a battery capable of operating for at least the entire life of the sensor. For example, the life of the sensor may be, for example, about 2 days, 3 days, 4 days, 5 days, 7 days, 10 days, 20 days, 30 days, 45 days, 60 days, a year, and the like. Alternatively, the life of the sensor may be shorter than 2 days, such as, about 36 hours, 30 hours, 24 hours, 12 hours, 6 hours, 3 hours and the like. The sensor power supply may be rechargeable. For example, the sensor power supply may be recharged when the sensor electronics are connected to the user interface. Additionally, the sensor power supply may be sized to last the entire duration that the sensor electronics are disconnected from the user interface, such as 15 minutes, 30 minutes, 1 hour, 2 hours, 3 hours, 4 hours, 6 hours, 8 hours, 12 hours, 24 hours, and the like. The sensor power supply may include one or more of a transformer, capacitor, power cell, solar cell, replaceable battery, and the like. Alternatively, the sensor power supply is a replaceable battery.

In the embodiment shown in FIG. 5C, the sensor electronics 120 include a sensor power supply 1210 and regulator 1090. Thus, when the sensor 100 is disconnected from the user interface 200, the sensor 100 remains powered. Because the sensor electronics do not include memory storage, the sensor data is not saved while the sensor 100 is not connected to the user interface 200.

As shown in FIG. 5E, it is possible to transport reference values with the sensor 100 so that the reference values are kept with the sensor 100 even when the sensor 100 is no longer connected to the user interface 200. In this embodiment, a sensor power supply 1210 and regulator 1090 and reference memory 1050 are included in the sensor electronics 120 that stay with the sensor 100 when disconnected from the user interface 200 at connector 400. When the sensor 100 is disconnected from the user interface 200, the sensor 100 may remain powered and retain calibration. Thus, the sensor 100 does not require re-calibration when re-connected. Furthermore, the sensor 100 may be connected to a different user interface and remain calibrated, because the calibration values are carried along with the sensor 100 and can be sent to the different user interface. If BG meter readings are needed for calibration, they are entered into the user interface 200 and sent to the reference memory 1050 in the sensor electronics 120. If BG meter readings are not needed, then the reference memory 1050 may contain factory installed reference values for the sensor. In the particular embodiment shown in FIG. 5E, sensor data is not collected while the sensor 100 is not connected to a user interface.

As shown in FIGS. 5D and 5F, the sensor electronics 120 may include a signal processor 1080. The signal processor simplifies communication across the tethered connection because the signal processor can convert weak analog sensor signals (which might be especially sensitive to noise) into digital signals, which can be made highly resistant to noise. Often, wires behave like antennas and gather radio frequency signals and the like, thus adding noise to signals carried on the wires.

As shown in FIGS. 5E-5H, the user interface 200 may be tethered to the sensor electronics 120, and the sensor electronics 120 may include a reference memory 1050. One or more reference values may be periodically measured, entered into the user interface 200 and transferred to the reference memory 1050, as shown in FIGS. 5E and 5G. If the sensor 100 is disconnected from the user interface 200 when a new reference value is required, the sensor 100 will need calibration when it is re-connected. As shown in FIGS. 5E and 5G, the power supply 1210, regulator 1090 and reference memory 1050 may be included with the sensor electronics 120. If the sensor 100 is disconnected from the user interface 200, the sensor 100 remains powered and retains calibration. Thus, the sensor does not require re-calibration or warm up when re-connected. Furthermore, the sensor may be disconnected from a first user interface and then connected to a second user interface and remain calibrated because the calibration values are carried along with the sensor and can be sent to the second user interface.

As shown in FIGS. 5E, and 5F, the sensor electronics 120 includes the reference memory 1050, sensor power supply 1210 and regulator 1090, but does not include the measurement memory 1060. Since the measurement memory 1060 is not included with the sensor electronics 120, the sensor data is not collected while the sensor 100 is not connected to a user interface. Furthermore, if periodic reference measurements are required, and the sensor electronics 120 are disconnected from the user interface 200 at the time that a new reference measurement is needed, then the sensor 100 will lose calibration, and a new reference measurement will be needed when the sensor electronics 120 are reconnected to a user interface.

As shown in FIG. 5G, the sensor electronics 120 may include the reference memory 1050, sensor power supply 1210, regulator 1090, signal processor 1080, measurement processor 1070, and the measurement memory 1060. Since the measurement memory 1060 is included with the sensor electronics 120, the sensor data is collected even while the sensor 100 is not connected to a user interface. Thus, a patient wearing a sensor may move about freely while disconnected from the user interface, and when they reconnect, all of the sensor data can be sent to the user interface for analysis and display. If however, periodic reference measurements are required, and the sensor electronics are disconnected from the user interface at the time that a new reference measurement is needed, then the sensor may lose calibration, and a new reference measurement will be needed when the sensor electronics are reconnected to a user interface.

Periodic reference values may not be required. One or more reference values may be stored in the reference memory at the factory. Furthermore, the reference memory may be non-volatile such as a flash memory, and therefore not require power to maintain the reference values as shown in FIG. 5H. Thus, reference values might be factory installed with each sensor and no power is required to maintain the reference values in the reference memory. As shown in FIGS. 5E, 5F, 5G and 5H, the reference memory 1050 may be included in the sensor electronics 120. Thus, a sensor may be disconnected from a user interface and connected to a second and not require calibration. The sensor may, however, require a warm up period if it loses power when disconnected from a user interface as shown in FIG. 5H.

Alternatively, one or more factory installed reference values may be stored in volatile memory with each sensor, and power is required to maintain the reference values in memory as shown in FIGS. 5E, 5F and 5G. The reference memory and a sensor power supply may optionally be included in the sensor electronics. Thus, a sensor may be disconnected from a user interface and connected to a second and not require calibration and the sensor may not require a warm up period if it does not lose power when disconnected from a user interface.

The tether may include one or more wires or one or more fiber optic cables or the like. Alternatively, the tether may not include a wire or cable or the like if the sensor electronics includes a sensor power supply and a regulator, and thus a wire is not needed to carry power to the sensor.

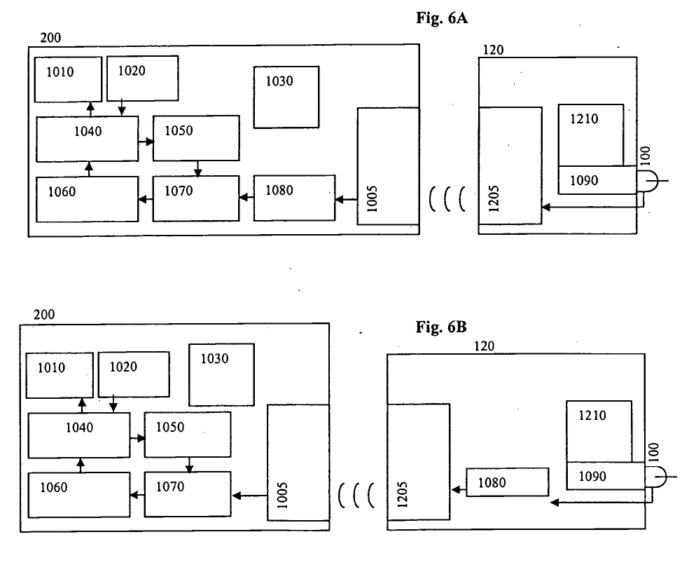

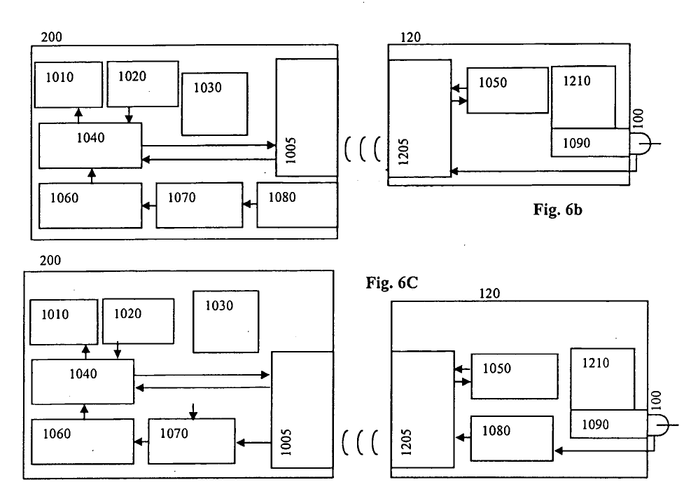

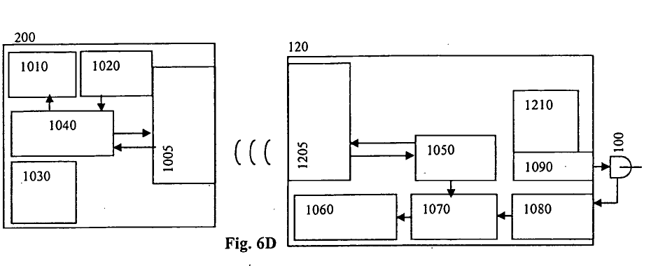

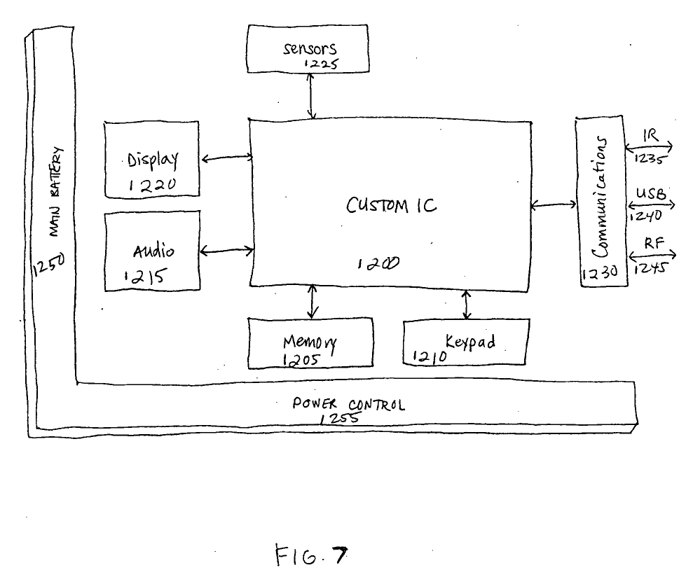

As shown in FIGS. 6A-6E, and as discussed above with respect to FIGS. 2A-2S and 4A-4C, the sensor electronics 120 may include a mechanism for wireless communication 1205, such as a radio frequency (RF) transmitter or transceiver, or an infrared (IR) transmitter or transceiver, light emitting diode (LED), sonic transmitter such as a speaker, and the like. Sensor electronics that include wireless communication capability are a subset of all sensor electronics and are referred to as wireless sensor electronics. Thus, a sensor may be physically coupled to wireless sensor electronics and establish a wired connection between the wireless sensor electronics and the sensor, but the wireless sensor electronics and sensor are not tethered to a user interface or an auxiliary device. Thus, a user can wear the sensor and move about freely, physically disconnect from other devices. This can be useful if the patient needs to leave the proximity of one or more devices. For example, if the patient is wearing a sensor with wireless sensor electronics that communicate with a stationary device such as a wall-mounted or bed-mounted display, then the patient may leave the room for a therapeutic procedure without having to disconnect the sensor electronics from any devices. Communication between the sensor electronics and one or more devices may be interrupted and may be re-established later. For example, the sensor electronics may be temporarily moved out of range for RF communication with a wall mounted device, or may be temporarily misaligned for IR communication with one or more devices.

The sensor wireless communication mechanism may be a processor that handles the communication protocol and manages transferring information in and out of the reference memory and the measurement memory. The measurement memory may contain one or more of calibrated measurements, time and dates associated with measurements, raw un-calibrated measurements, diagnostic information, alarm history, error history, settings and the like. Settings may be determined by a user using a keypad on the user interface, and the settings are sent to a memory in the sensor electronics. Additionally, the sensor wireless communication mechanism may be a processor that evaluates the calibrated measurements according to user defined settings and sends results of the evaluation to the user interface. For example, the user may set an alarm threshold, which is sent to be stored in a memory in the sensor electronics. Then the sensor wireless communication mechanism compares a calibrated measurement to the alarm threshold and if the calibrated measurement exceeds the alarm threshold, the communication system sends an alarm message to the user interface. Finally, the user interface displays the alarm message.

The alarms may function even when the sensor and sensor electronics are disconnected from the user interface and/or patient monitor. In this way, the patient will be warned if he/she becomes hyperglycemic or hypoglycemic, even when not connected to the user interface and/or patient monitor. For example, the sensor electronics may be coupled to an alarm. As discussed above, an alarm threshold may be stored in a memory in the sensor electronics. If a calibrated measurement exceeds the alarm threshold, the alarm coupled to the sensor electronics may be activated. Similarly, if a battery is low on power, or the sensor is not performing properly, or communication with another device has been lost, or an error has occurred, or a warning is needed, then the sensor electronics may activate an alarm. The alarm may be an audible alarm, a visible alarm, a tactile alarm (such as a vibrating alarm), or any combination thereof. In particular embodiments, the sensor electronics includes one or more components for alarming a user

User defined parameters such as alarm thresholds, minimum time between alarms, alarm snooze time, trend alarm thresholds, patient ID, one or more identifying codes, a password, and the like may be sent from the user interface to the sensor electronics and stored in memory in the sensor electronics. Thus, settings that are established for a particular patient are not lost when the patient is moved to a new location and the sensor electronics establishes communication with a second user interface. The user defined settings are sent the second user interface when communication is first established with sensor electronics. Each set of sensor electronics may have a unique ID, code, name, serial number, or the like, which is sent to the user interface so that the user interface can identify which sensor electronics it is communicating with. The unique ID for a sensor electronics may be required to be entered into a user interface before the user interface will recognize communications from a sensor electronics. Thus, if a user interface detects communication from more than one sensor electronics, then user interface can determine which signal to respond to based on the unique ID contained in the communications. Furthermore, the user interface and/or auxiliary devices may have one or more unique IDs so that each device, user interface, and sensor electronics can determine whether to accept communications from each other. For example, a patient monitor may be programmed to accept communications from a user interface or sensor electronics as long as the communication includes a unique ID representing a particular sensor. Thus, if two patients share a room and transmissions from a first patient's sensor electronics are received by a second patient's user interface and/or patient monitor, the second patient's user interface and/or patient monitor will ignore the communication. Yet, the first patient's user interface and/or patient monitor will accept the communication from the first patient's sensor electronics. In another example, a user interface ID number is entered into a patient monitor, and the patient monitor will only accept communications that contain the user interface ID number.

As discussed above, alarms may be provided for a number of desired conditions. For example, alarms or other alerts may be provided when a user's glucose level is approaching a predefined threshold, or has exceeded a predefined threshold, which may indicate that a user is approaching hypo- or hyper-glycemia. An alarm may be triggered by change in trends of analyte levels or by the current value of an analyte level. The alarm may be activated when a specific bolus amount is required to be dispensed. The alarm may indicate that an occlusion has occurred in a pump or that the syringe portion of a syringe-type infusion pump is not seated properly. The alarm may be an audio, visual, and/or tactile alarm. For an audible alarm, such as beeping, the alarm may get increasingly louder. For a tactile alarm, such as a vibration, the alarm may get increasingly stronger and/or faster. For a visual alarm, such as flashing or changing of color or indication of an alarm by an icon, the alarm may get increasingly brighter, faster, and/or larger. A visual alarm may also be conducted through SMS text messages on the monitor. In embodiments, the alarm may have a snooze option. In further embodiments, the alarm is through mp3's or system tones, such as beeping. In still further embodiments, the alarm is a personalized voice tag alarm, in which a parent, physician, caretaker, or other person may record a warning that plays upon activation (e.g. “your blood glucose is low,” “you need to take a bolus,” etc.).

The alarms may be customized to specific user needs. The alarm may be set to flashing lights for the hearing impaired, or warning sounds and/or vibration for the vision impaired. There could further be included headphones that can plug into the analyte sensor for vision impaired to instruct the user on what to do in the case that an alarm goes off. The headphones could also be plugged into a MPEG player or the like.

In other embodiments, a speaker is included to provide an alternative mode of communication. In an embodiment, the analyte sensor, such as a BG sensor, may use the speaker to announce a message that states “move nearer to pump” when the sensor senses that the communication with the medication device is weak or interrupted. In the alternative, the analyte sensor may simply display a text message that states “move nearer to pump.” A similar message may be displayed if the BG sensor senses some type of problem or malfunction. Alternatively, an alarm may alert the user of any problem or malfunction by vibrating, emitting warning sounds, flashing light, and the like.

FIGS. 6A-6E show similar embodiments to FIGS. 5A-5H. However, as shown in FIGS. 6A-6C, the sensor electronics 120 include sensor wireless communication mechanism 1205 and the user interface 200 includes user interface wireless communication mechanism 1005. As shown in FIG. 6A, the sensor power supply 1210 and regulator 1090 are part of the sensor electronics 120. Thus, the sensor 100 constantly remains powered. As shown in FIG. 6B, the signal processor 1080 may reside in the sensor electronics 120, so that the sensor 100 can remain powered but can also perform processing. In particular embodiments, if the signal processor 1080 includes an analog to digital converter. Thus, digital communication can be used to send the processed sensor signal to the user interface 200.

Once the sensor is powered and warmed up by the sensor power supply and the regulator, the sensor remains powered and sufficiently warmed up and thus does not need to warm up again no matter how many different devices it communicates with. One or more reference values may be measured periodically and stored in the reference memory when they are collected. If the wireless sensor electronics cannot establish communication with user interface when a new reference value is required, the sensor will need calibration when communication is re-established.

As shown in FIG. 6C, the sensor power supply 1210, regulator 1090 and reference memory 1050 may stay with the sensor 100. Then if the sensor 100 loses communication with the user interface 200 (such as because the patient walks too far away from the user interface), then the sensor remains powered and retains calibration. Thus, the sensor 100 does not require re-calibration or warm up time when it re-establishes communication with the user interface 200. Furthermore, the sensor 100 may establish communication with a second user interface and remain calibrated because the calibration values are carried along with the sensor 100 and can be sent to the second user interface. As shown in FIG. 6D, the wireless sensor electronics may include the reference memory 1050, sensor power supply 1205, regulator 1090, signal processor 1080 and a wireless communication mechanism 1205, but does not include the measurement memory 1060. Since the measurement memory is not included with the wireless sensor electronics, the sensor data is not collected while the wireless sensor electronics is not in communication with a user interface. Furthermore, if periodic reference measurements are required, and communication cannot be established between the wireless sensor electronics and the user interface at the time that a new reference measurement is needed, then the sensor will lose calibration, and a new reference measurement will be needed when the wireless sensor electronics and a user interface have established communication.

As shown in FIG. 6E, in addition to the sensor power supply 1210, regulator 1090, reference memory 1050, the measurement memory 1070 and measurement processor 1060 may stay with the sensor 100. When communication is lost between the sensor electronics 120 and the user interface 200, the sensor 100 remains powered, retains calibration and collects and stores measurements. Thus, the sensor 100 does not require re-calibration or warm up when communication is established with any user interface. A patient wearing a sensor may move about freely, and when the wireless sensor electronics establishes communication with a user interface all of the sensor data can be sent to the user interface for analysis and display. If however, periodic reference measurements are required, and the wireless sensor electronics and user interface cannot establish communication at the time that a new reference measurement is needed, then the sensor may lose calibration, and a new reference measurement will be needed when the wireless sensor electronics are in communication with a user interface.

Alternatively, periodic reference values are not required. One or more reference values may be stored in the reference memory at the factory. Furthermore, the reference memory may be non-volatile such as a flash memory, and therefore not require power to maintain the reference values. Thus, reference values might be factory installed with each sensor and no power would be required to maintain the reference values in the reference memory. The reference memory may be included in the wireless sensor electronics. Thus, calibration would not be required when the sensor electronics establishes communication with a user interface.

Alternatively, one or more factory installed reference values may be stored on a volatile reference memory in wireless sensor electronics that are included with each sensor. In this case, power could be needed to maintain the reference values in memory. Alternatively, the reference memory and a sensor power supply are included in the wireless sensor electronics.

If the reference values are factory installed, they may be included on a CD, floppy disk, or other removable storage devices. If the reference values are stored on a CD, for example, they may be downloaded into a personal computer and then downloaded into the user interface and/or sensor electronics. The reference values may also be stored on a removable or non-removable non-volatile memory. For example, if the reference values are stored on a removable non-volatile memory, the memory may be included in a flash memory card. The flash memory card may be adapted to be used in the user interface and/or the sensor electronics. The reference values may be stored on a non-volatile or volatile memory that is included with the sensor electronics at the factory. In this case, if the memory included with the sensor electronics is volatile, the sensor electronics should include a power source so that the sensor electronics may retain the reference values during shipping and storage. One set of sensor electronics may contain reference values to calibrate a number of sensors. For example, if a sensor electronics is shipped with a number of sensors, the reference values may calibrate all of those sensors.

The user interface and/or the sensor electronics may include a slot for a flash memory card. The flash memory card may include reference values that are factory input or reference values that are input later. Additionally, the flash memory card may store additional desired data. The flash memory card may be included when the user interface and/or sensor electronics is shipped from a factory or reseller. Or, the flash memory card may be purchased separately for use with the user interface and/or the sensor electronics. Additionally, a flash memory card may be used in the patient monitor.

As noted above with respect to FIGS. 6C, 6D, and 6E, the wireless sensor electronics 120 may include a reference memory 1050. One or more reference values may be periodically measured, entered into the user interface and sent to the reference memory 1050. If communication cannot be established between the wireless sensor electronics 120 and the user interface 210 when a new reference value is required, the sensor 100 will need calibration when it is re-connected. Alternatively, reference measurements are sent directly to the wireless sensor electronics 120. Some examples include: a BG meter with an IR transmitter sends a reference measurement to the wirelesses sensor electronics which include an IR receiver; a BG meter with RF communication capability sends a BG value to a wireless sensor electronics with an RF receiver; and a laboratory analyte measurement machine analyzes a blood sample and the result of the analysis is sent to an RF transmitter which transmits the result to the wireless sensor electronics.

FIG. 7 shows an electronics architecture according to an embodiment of the invention with a custom integrated circuit (“custom IC”) 200 as the electronics processor. This architecture can support many of the devices discussed herein, for example the analyte sensor, the medication device, the controller device, or any combination of the above. The custom IC 1200 is in communication with a memory 1205, keypad 1210, audio devices 1215 (such as speakers or audio electronic circuitry such as voice recognition, synthesis or other audio reproduction), and a monitor or display 1220. The custom IC 1200 is in communication with the sensor 1225 included in the device, or in communication with the device (for example, a BG sensor or a device which includes an analyte determining function). The electronics architecture further may include a communications block 1230 in communication with the custom IC 1200. The communications block 1230 may be adapted to provide communication via one or more communications methods, such as RF 1235, a USB 1240, and IR 1245. In further embodiments, the custom IC 1200 may be replaced by electronic circuitry, discrete or other circuitry, with similar functions.

The electronics architecture may include a main battery 1250 and a power control 1255. The power control 255 may be adapted to give an end of battery warning to the user, which can be predicted based on the type of battery used or can be calculated from the power degradation of the battery being used. However, in certain embodiments it is not necessary to know the type of battery used to create an end of battery warning. Various battery types, such as rechargeable, lithium, alkaline, etc., can be accommodated by this design. In certain embodiments, the electronics architecture includes a removable battery and an internal backup battery. Whenever a new removable batter is inserted, the internal backup battery will be charged to full capacity and then disconnected. After the removable battery has been drained of most of its energy, it will be switched out of the circuit and the internal backup battery will be used to supply power to the device. A low battery warning may then be issued. The internal backup battery may be rechargeable. In further embodiments, a supercap, for example, is used to handle the peak loads that the rechargeable internal battery could not handle directly, because it has sufficient energy storage. This method also allows the use of any type of removable battery (alkaline, lithium, rechargeable, etc.) and partially drained batteries. Depending on use, the backup battery may allow the device to operate for at least one day after the removable battery has been drained or removed. In further embodiments, a microprocessor measures the charge states and control switches for removable and internal backup batteries.

Alternatively to the types of memory discussed above, a removable nonvolatile reference memory may be filled at the factory with reference values for calibrating one or more sensors. The removable nonvolatile reference memory may be a flash media such as a flash card, memory stick, and the like. The reference memory may placed into the user interface and/or into the sensor electronics. The removable nonvolatile reference memory may be placed into a device such as, an auxiliary device, a meter, a BG meter, a palm pilot, a phone, a PDA, a handheld device, a patient monitor, a module that connects to a device, and the like. If a new sensor cannot be calibrated with a removable nonvolatile reference memory that is presently in a device, then the sensor will be accompanied with a new removable nonvolatile reference memory for use in a device.

An auxiliary device may provide power to a user interface, which in turn powers the sensor. The user interface may have a rechargeable power source that provides power to the user interface whenever power is not supplied by the auxiliary device. For example, an auxiliary device such as a patient monitor may provide power along a wire through a connector to a user interface; the user interface has a power supply; a sensor is connected by a wire to the user interface; the power from the auxiliary device powers a voltage regulator in the user interface, which powers the sensor. If the user interface is disconnected from the auxiliary device, the user interface power supply continues to supply power to the sensor. Alternatively, the auxiliary device may charge the user interface power supply whenever the auxiliary device is connected to the user interface, and the user interface may power the sensor whether or not the auxiliary device is connected to the user interface.

As shown in FIG. 8, in further embodiments, the sensing device 2000, which includes the sensor 2100, for example, a blood glucose sensor, and sensor electronics 2120 may be adapted to interact with an auxiliary device 2300. In particular embodiments, the auxiliary device 2300 is a hospital monitor. Although the sensing device 2000 is shown as having the sensor 2100 attached to the sensor electronics 2120, they may be wired or otherwise coupled together or may be within the same housing, as discussed above. Also as discussed above, transmission may be wired or wireless. As shown in FIG. 8, the sensing device is sensing analyte data 2250, such as blood glucose data. The sensing device 2000 is adapted to transmit device information 2600 to the auxiliary device 2300. The auxiliary device 2300 is adapted to transmit requests for device information 2500 to the sensing device 2000. Both transmissions may occur while the sensing device is sensing data. For example, in a hospital setting, it is not necessary to remove the sensing device from the patient to transmit data to the hospital monitor. Device information may include any of the information discussed herein as being stored in the sensor, for example, patient data such as patent identifications, sensor data such as sensor identification, previously or currently sensed analyte data, calibration data, historical data, alarm data, and so forth.

In further embodiments, the auxiliary device transmits requests for device information to the sensing device, in response to which the sensing device may automatically transmit the requested information without further interaction from a user. The auxiliary device is adapted to receive communications from the sensor whenever a patient moves into a new room. A receiver on an auxiliary device, such as a hospital monitor, is adapted to receive communications from sensors. When the patient is moved to the new room, all of the information stored in the sensor electronics may automatically be displayed on the hospital monitor. There may be a predetermined distance within which the sensing device needs to be from the hospital monitor for transmission to begin. Where the patient has been away from a monitor or other auxiliary device for a long time, then it is beneficial to have the sensed data stored in the sensor electronics so that no data is missed merely because the patient has been away from an auxiliary device. In further embodiments, the sensing device may periodically transmit ready communications wireless, to indicate that it is looking for a hospital monitor or other auxiliary device and is ready to transmit data. “Periodically” may mean once a predetermined number of minutes (such as 1 or 5) or seconds or may mean continuously. When the hospital monitor receives the ready communication, it sends a transmission to the sensing device requesting that the sensing device send over device information. The hospital monitor may request that the sensing device send some or all of the data stored in and/or currently being measured by the sensing device.

In further embodiments, the sensor may include a method of notifying the auxiliary device that the sensor is leaving the transmission area. For example, a wireless sensor may be coupled to a button that, when pressed, sends a transmission to the auxiliary device indicating that the sensor is leaving the transmission area. Thus, the auxiliary device will stop searching for sensor transmissions. Alternatively, the user may input into the auxiliary device a request to stop searching for the sensor. In further embodiments, the auxiliary device includes an key, button, or other input to indicate that the auxiliary device should start or stop searching for a sensor. In still further embodiments, the auxiliary device may interact with a wand, such as a magnetic wand, that when passed over a portion of the auxiliary device is adapted to “wake up” the auxiliary device receiver to look for transmissions. The auxiliary device may further be adapted to receive the identifying information such as an identification number of a sensor, for example from a keypad or directly from the sensor. The auxiliary device may then be adapted to transmit a message to the sensor, requesting that it indicate that the sensor has been properly detected and that the sensor and auxiliary device are in communication. For example, the sensor may be equipped with an audible device like a speaker (which may beep or make another sound), a visible device like a light or screen (which may flash or pop up an icon, for example), and/or a tactile device like a vibrator that will make a vibration. Any of these devices may indicate that the sensor has been properly reached by the auxiliary device transmission. Alternatively, or in addition, the sensor may send a transmission back to the auxiliary device indicating that it was properly reached. The auxiliary device may then display, sound, or otherwise indicate that the sensor is now in communication with the auxiliary device.

In further embodiments, identification information transferred between the auxiliary device and the sensor may include patient identification data, for example, patient ID number, name, or the like. The patient identification data may be entered from the monitor or directly into the sensor device through the user interface. In certain embodiments, the patient identification information is transmitted with every transmission to the auxiliary device. It is common for hospitals to have electronic data-management systems. By sending patient identification information with transmissions, the sensed data being transmitted can be automatically entered into the patient's electronic file. Also, the inclusion of patient information allows monitors and auxiliary devices in other parts of the hospital to more easily sync up with the sensor.

In hospitals, the same sensor electronics may be used for a number of different patients. When a new sensing element is connected to the sensor electronics for start-up, in certain embodiments, the user interface displays a request to the user to ask whether the patient is a new patient. If the user indicates that the patient is a new patient, the memory with the old patient history can be cleared. In further embodiments, the user has the option to retain the old patient history.

In further embodiments, the sensor includes reminders. These reminders may, for example, be reminders that it is time to administer a drugs or another therapy to the patient or reminders that it is time to take blood pressure or administer another test. The sensor may also have warnings to indicate to the user that certain therapies and drugs should not be administered. These could be based on a preprogrammed or downloaded library or based on data input by a doctor or other user. For example, a doctor may input that a particular drug should not be administered to the patient, for allergy or drug interaction reasons. If the sensor is adapted to receive information about the different drugs being administered to the patient, when the nurse checks the sensor, it will warn the nurse not to administer that drug.

The sensor may be powered by sensor electronics, which are powered by a device such as an auxiliary device or a user interface. The sensor electronics may have a rechargeable power supply that keeps the sensor powered whenever power is not supplied by a device.

The power needed to operate a sensor may be generated at a device such as a user interface or an auxiliary device, carried over one or more wires, passed through a transformer and supplied to the sensor. Alternatively, the power may be passed through a regulator such as a voltage regulator and a current regulator before it is supplied to a sensor. The transformer may be located in the device or the transformer may be part of the wire or cable connecting the sensor to the device. The transformer also may be in the sensor electronics. The transformer keeps the sensor powered as long as the sensor is connected to the device. The transformer helps to remove a ground connection between the device and the sensor, and therefore isolates the patient from the ground voltage in the device.

The sensor signal may be passed to one or more devices before it is processed. For example, the sensor signal could be carried along a wire to a user interface, and then carried along a wire to an auxiliary device before it is processed. In another example, the sensor signal is carried to a computer, sent through a server or a router to a second computer, and then processed.

The user interface may process the sensor measurements to generate insulin delivery commands. The insulin delivery commands may be infusion rates. Alternatively, the insulin delivery commands may be insulin amounts.

An auxiliary device may process the sensor measurements to generate insulin delivery commands. Alternatively, sensor electronics may process the sensor measurements to generate insulin delivery commands.

Further examples include giving the analyte sensor and/or user interface cellular telephone, pager or watch capabilities. These embodiments integrate commonly used devices with the analyte sensor so that the user may have one less device to carry. For example, the sensor housing may be integrated with the user interface and may include time-telling functions. For example, the sensor may be a wrist-worn device, such as a watch. The watch may include a credit card-sized display to facilitate easier viewing and adapted to display a time. The display of the time may be digital or analog. The time may be changed by the user using input devices like keys or buttons or a scroll wheel, depending on the set-up of the watch device. The watch display may be used to indicate the analyte levels, such as that of the user's glucose level. A watch having the above features is disclosed in U.S. patent application Ser. No. 11/496,606 entitled “Watch Controller for a Medical Device,” filed on Jul. 31, 2006, which is hereby incorporated by reference in its entirety. The sensor may also be a watch that can be carried on other parts of the body or clothing, such as the ankle, neck (e.g., on a chain), pocket, or ankle. Other options for integrating with the sensor include but are not limited to a key fob, PDA's, smart phones, watch remotes, and the like. The analyte sensor may further communicate with, and download data such as software upgrades and diagnostic tools from, a remote station like a computer from a connector.

The insulin delivery commands may be generated in the device that contains the measurement processor. Alternatively, the insulin delivery commands may be generated by a device that receives sensor measurements, such as an auxiliary device, a pump, and the like. Still alternatively, the insulin delivery commands are generated by an insulin infusion pump such as shown in U.S. Pat. Nos. 4,562,751, 4,678,408, 4,685,903, 5,080,653, 5,097,122, and 6,554,798, which are herein incorporated by reference.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A sensing device for monitoring blood glucose concentration of a patient, the sensing device comprising:
a blood glucose sensor to sense blood glucose data,
sensor electronics in electrical communication with the blood glucose sensor, adapted to communicate with a hospital monitor including (a) transmitting device information to the hospital monitor while the sensor is sensing the blood glucose data; and (b) transmitting sensed blood glucose data to the hospital monitor.

2. The sensing device of claim 1, wherein the transmitting of device information to the hospital monitor is automatic when a request is received from the hospital monitor for the device information.

3. The sensing device of claim 1, wherein the transmitting of device information to the hospital monitor is in response to a request received from the hospital monitor.

4. The system of claim 1, wherein the sensor electronics periodically transmit via a wireless method a ready communication indicating that it is ready to communicate with a hospital monitor.

5. The sensing device of claim 1, wherein the device information includes patient information.

6. The sensing device of claim 6, wherein the patient information includes a patient identification number.

7. The sensing device of claim 1, wherein the device information includes a sensor identifying code, wherein the sensor electronics are adapted to transmit the identifying code to a hospital monitor.

8. The sensing device of claim 1, wherein the device information includes a history of sensed blood glucose data.

9. The sensing device of claim 8, wherein the history of sensed blood glucose data includes blood glucose data sensed during the previous forty minutes.

10. The sensing device of claim 1, further including an indicator to indicate that the hospital monitor is requesting device information from the sensing device.

11. The sensing device of claim 10, wherein the indicator is selected from an audible beep and a visual flash.

12. The sensing device of claim 1, wherein the communication between the sensor electronics and the hospital monitor is wireless.

13. The sensing device of claim 1, wherein the sensor electronics include a wireless transceiver for wireless communication between the hospital monitor and the sensor electronics.

14. The sensing device of claim 1, wherein the sensor electronics include a memory for storing blood glucose data sensed by the sensor.

15. The sensing device of claim 14, wherein the sensor electronics memory stores at least the previous four hours of sensor data.

16. The sensing device of claim 14, wherein the memory is nonvolatile.

17. The sensing device of claim 16, wherein the memory is a flash memory.

18. The sensing device of claim 14, wherein the memory is adapted to store calibration values.

19. The sensing device of claim 18, wherein the calibration values are values obtained from a blood glucose meter.

20. The sensing device of claim 19, wherein the blood glucose meter is coupled to the sensor electronics.

21. The sensing device of claim 19, wherein the blood glucose meter is coupled to the hospital monitor.

22. The sensing device of claim 21, wherein the blood glucose meter and the hospital monitor are integrated into a single housing.

23. The sensing device of claim 18, wherein the calibration values are values obtained from a laboratory test.

24. The sensing device of claim 1, wherein the sensor is a subcutaneous sensor.

25. The sensing device of claim 1, wherein the sensor is a real-time sensor to sense blood glucose data in real-time.

26. The sensing device of claim 1, wherein the sensor electronics include a processor to calibrate the blood glucose data and a calibration memory having calibrated blood glucose data stored therein.

27. A system for monitoring blood glucose concentration of a patient, the system comprising:
a sensing device including:
a blood glucose sensor to sense blood glucose data, and
sensor electronics in electrical communication with the blood glucose sensor; and
a hospital monitor adapted to communicate with the sensing device, wherein the sensor electronics are adapted to transmit device information to the hospital monitor while the sensor is sensing the blood glucose data and to transmit sensed blood glucose data and wherein the hospital monitor is adapted to send requests for device information to the sensor electronics.

28. The system of claim 27, wherein the transmission of device information to the hospital monitor is automatic when a request is received from the hospital monitor for the device information.

29. The system of claim 27, wherein the hospital monitor transmits the request when the sensing device is connected to the hospital monitor via wire.

30. The system of claim 27, wherein the hospital monitor transmits the request when the sensing device is within a predetermined distance from the hospital monitor.

31. The system of claim 27, wherein the sensing device periodically transmits via a wireless method a ready communication indicating that it is ready to communicate with a hospital monitor, and the hospital monitor transmits the request when the hospital monitor receives the ready communication.

32. The system of claim 27, wherein the hospital monitor is adapted to monitor at least one parameter other than blood glucose.

33. The system of claim 27, wherein the device information includes patient information.

34. The system of claim 27, wherein the device information includes a sensor identifying code, wherein the sensor electronics are adapted to transmit the identifying code to the user interface, and wherein the hospital monitor is adapted to identify the sensor based on the identifying code before storing data from the sensor.

35. The system of claim 27, wherein the device information includes a history of sensed blood glucose data.

36. The system of claim 35, wherein the device information includes calibration history of the sensing device.

37. The system of claim 27, wherein the sensing device further includes an indicator to indicate that the hospital monitor is requesting device information from the sensing device.

38. The system of claim 27, wherein the hospital monitor and the sensing device each include a wireless transceiver for wireless communication between the hospital monitor and the sensing device.

39. The system of claim 27, wherein the sensor electronics include a memory for storing blood glucose data sensed by the sensor.

40. The system of claim 27, wherein the memory is a flash memory.

41. The system of claim 27, wherein the memory is adapted to store calibration values.

42. The system of claim 41, wherein the calibration values are values obtained from a blood glucose meter.

43. The system of claim 42, wherein the blood glucose meter is coupled to the sensor electronics.

44. The system of claim 42, wherein the blood glucose meter is coupled to the hospital monitor.

45. The system of claim 44, wherein the blood glucose meter and the hospital monitor are integrated into a single housing.

46. A method of monitoring blood glucose values of a patient, the method comprising:
sensing blood glucose data from a sensing device including a blood glucose sensor and sensor electronics in communication with the blood glucose sensor;
transmitting device information from the sensing device to a hospital monitor while sensing the blood glucose data; and
transmitting sensed blood glucose data to the hospital monitor.

47. The method claim 46, further comprising receiving a request from a hospital monitor for device information, wherein the transmitting device information is automatic in response to the request.

48. The method of claim 46, further comprising periodically transmitting via a wireless method a ready communication indicating that the sensing device is ready to communicate with a hospital monitor.

49. The method of claim 46, wherein the transmission of device information is a wireless transmission.

50. The method of claim 46, wherein the sensor electronics include a memory and the method further comprises storing the sensed blood glucose data in a memory.

51. The method of claim 50, further comprising storing calibration values in the memory.

52. The method of claim 46, wherein the sensor is a subcutaneous sensor.
